(19) United States
(12) Patent Application Publication
(10) Pub. No.: US 2021/0318306 A1
(43) Pub. Date: Oct. 14, 2021

(54) Compositions for and Methods of Neutralizing Lipopolysaccharide Toxicity and Methods of Identifying the Same

(71) Applicant: Colgate-Palmolive Company, New York, NY (US)

(72) Inventors: Dandan Chen, Bridgewater, NJ (US); Harsh Mahendra Trivedi, Hillsborough, NJ (US); James Masters, Ringoes, NJ (US)

(73) Assignee: Colgate-Palmolive Company, New York, NY (US)

(21) Appl. No.: 17/301,428

(22) Filed: Apr. 2, 2021

Related U.S. Application Data
(60) Provisional application No. 63/004,174, filed on Apr. 2, 2020.
(60) Provisional application No. 63/200,318, filed on Mar. 1, 2021.

(51) Int. Cl. G01N 33/566; G01N 33/543; A61K 33/30; A61K 33/16; G01N 33/50; A61P 31/04

(52) U.S. Cl. CPC G01N 33/566; G01N 33/54326; A61K 33/30; A61K 33/16; G01N 2400/50; A61P 31/04; G01N 33/5091; G01N 2333/195; G01N 2333/705; G01N 33/5041

(57) ABSTRACT

Methods for identifying a composition that neutralizes toxicity of a lipopolysaccharide are disclosed. The methods comprise comparing the amount of cytokine and/or prostaglandin E2 secreted by cells that have Toll-like receptors activated by a lipopolysaccharide in the presence or absence of a test composition. Methods of neutralizing toxicity of a lipopolysaccharide in an individual's oral cavity using compositions identified neutralizing toxicity of a lipopolysaccharide are also disclosed. Method of neutralizing toxicity of a lipopolysaccharide in an individual's oral cavity using oral care compositions that comprise combinations of zinc oxide and zinc citrate are also disclosed.

### CROSS-REFERENCE TO RELATED APPLICATIONS

This application claims priority to U.S. Provisional Application No. 63/004,174 filed Apr. 2, 2020, which is incorporated herein by reference in its entirety, and U.S. Provisional Application No. 63/200,318 filed Mar. 1, 2021, which is incorporated herein by reference in its entirety.

### BACKGROUND

Oral bacteria are highly associated with oral diseases. Many major perio-pathogens can coexist as a normal flora in the host. Over time, these bacteria can increase their numbers, and induces chronic inflammation. Plaque is the soft, sticky, colorless film of bacteria that forms constantly on the teeth and gums. If plaque is not removed by daily brushing and flossing, it accumulates and hardens over time. Left untreated, plaque leads to inflammation or infection of the gum tissue associated with gingivitis. Untreated gingivitis can eventually spread from the gums to the ligaments and bone that support the teeth, and develop into periodontitis. The inflammation of the tissues around the tooth due to accumulation of dental plaque is considered the main characteristic of acute and chronic periodontitis. Chronic periodontal inflammation can cause the teeth loss as an outcome destroying the alveolar bone.

Plaque results in an increased number of Gram-negative bacteria. The pathogenic bacteria in plaque produce a virulent endotoxin, lipopolysaccharide (LPS), that causes the chronic inflammation with of the gum line which can progress to affect the bone that surrounds and supports teeth. LPS is a large, complex molecule that is a major component of the outer membrane of Gram-negative bacteria. LPS released from Gram-negative cell wall functions as an endotoxin and stimulates immune responses that result in inflammation. LPS from different strains of gram-negative bacteria differ but share a common structural pattern. The components of an LPS include a lipid A fraction, a core polysaccharide region composed of an inner core and an outer core, and an O-antigen. The differences among the structures of LPSs result in their different virulence, i.e. levels of endotoxicity. Thus, some LPS are more harmful than others.

LPS released in the oral cavity activates Toll-like receptors on the surface of cells within the oral cavity. Bacterial LPS binds the TLR4 receptor which then dimerizes and initiates a kinase cascade culminating in nuclear activation of NFκB. The activated Toll-like receptor mediates an NF-κB signaling pathway that induces the cell to release critical proinflammatory cytokines necessary to stimulate potent immune responses that lead to tissue destruction. This signals production of various pro-inflammatory cytokines, including interleukin 8 (IL-8), tumor necrosis factor alpha (TNFα), and prostaglandin E 2 (PGE 2). Higher levels of the cytokines correlate with higher levels of bacterial LPS and indicate more inflammation. Because periodontal diseases are associated with chronic inflammation due to oral bacteria, bacterial LPS-induced inflammation is a direct cause of periodontal disease. Porphyromonas gingivalis LPS stimulation of TLR4 results in an increase in IL-8 and TNFα, and this increase is linked to periodontal disease.

### BRIEF SUMMARY

Methods for identifying a composition that neutralizes toxicity of a lipopolysaccharide are provided. The methods comprise performing a test assay, performing a control assay and comparing the results of each to each other to determine whether a test composition neutralizes toxicity of a lipopolysaccharide. The test assay is performed by contacting, in the presence of a test composition, a Toll-like receptor reporter cell with an amount of a lipopolysaccharide sufficient to stimulate secretion of one or more proinflammatory cytokines and/or prostaglandin E2 by the Toll-like receptor reporter cell, and measuring the amount of one or more proinflammatory cytokines and/or prostaglandin E2 secreted by the Toll receptor reporter cell in the test assay. The control assay is performed by contacting, in the absence of the test composition, the Toll-like receptor reporter cell with the amount of the lipopolysaccharide sufficient to stimulate secretion of one or more proinflammatory cytokines and/or prostaglandin E2 by the Toll-like receptor reporter cell, and measuring the amount of one or more proinflammatory cytokines and/or prostaglandin E2 secreted by the Toll-like receptor reporter cell in the control assay. The amount of one or more proinflammatory cytokines and/or prostaglandin E2 secreted by the Toll-like receptor reporter cell in the test assay is compared with the amount of one or more proinflammatory cytokines and/or prostaglandin E2 secreted by the Toll-like receptor reporter cell in the control assay. If the amount of one or more proinflammatory cytokines and/or prostaglandin E2 secreted by the Toll-like receptor reporter cell in the test assay is less than the amount of one or more proinflammatory cytokines and/or prostaglandin E2 secreted by the Toll-like receptor reporter cell in the control assay, the test composition is identified as a composition that neutralizes toxicity of a lipopolysaccharide.

In various embodiments, the Toll-like receptor reporter cell may be a recombinant eukaryotic cell that expresses one or more Toll-like receptors, such as for example, a recombinant HEK 293T cell, a recombinant human monocyte cell or a recombinant Chinese Hamster ovary cell. In various embodiments, human gingival tissue is used in assays and a human gingival cell of the human gingival tissue is the Toll-like receptor reporter cell. Human gingival cells of the human gingival tissue comprise one or more Toll-like receptors and may be recombinantly transformed to produce cells that express one or more additional Toll-like receptors. Recombinant cells express one or more Toll-like receptors such as TLR1, TLR2, TLR3, TLR4, TLR5, TLR6, TLR7, TLR8, TLR9 and TLR10. The lipopolysaccharides used are generally from pathogenic gram-negative bacteria, such as for example, Porphyromonas gingivalis, Escherichia coli, Prevotella intermedia, Fusobacterium nucleatum, Treponema denticola, Aggregatibacter actinomycetemcomitans or Tannerella forsythia. Other pathogenic bacteria, may include for example, Eikenella corrodens, Campylobacter rectus, Campylobacter gracilis, Streptococcus mutans, Streptococcus sobrinus, Streptococcus sanguis, Streptococcus oralis, Actinomyces israelii, Chlamydia pneumoniae, Porphyromonas cangingivalis, Fusobacterium necrophorum, and Streptococcus constellatus. Activation of the TLR by the LPS stimulates the TLR reporter cells to secrete proinflammatory cytokines and/or prostaglandin E2 that can be detected and measured. Proinflammatory cytokines that can be measured include TNF-α, IL-6, IL-8, IL-1β and GM-CSF. In some embodiments, multiple proinflammatory cytokines are measured. In some embodiments, proinflammatory cytokine-specific magnetic beads are used in the assays to measure the amount of proinflammatory cytokines secreted. In some embodiments, prostaglandin E2 is measured. In some embodiments, one or more proinflammatory cytokines and prostaglandin E2 are measured. In some embodiments, multiple proinflammatory cytokines and prostaglandin E2 are measured.

The test composition is identified as a composition that neutralizes toxicity of a lipopolysaccharide may be used in methods of neutralizing toxicity of a lipopolysaccharide in an individual's oral cavity. Such methods that are include those that comprise administering to the oral cavity of the individual an oral care composition comprising such compositions that neutralizes toxicity of the lipopolysaccharide. The compositions are applied to the individual's oral cavity in an amount effective to inhibit secretion of one or more proinflammatory cytokines and/or prostaglandin E2 by cells of the individual.

Method of neutralizing toxicity of a lipopolysaccharide in an individual's oral cavity comprising administering to the oral cavity of the individual an oral care composition comprising zinc oxide and zinc citrate are also provided. The oral care composition comprises zinc oxide and zinc citrate in an amount effective to inhibit secretion of one or more proinflammatory cytokines and/or prostaglandin E2 by cells of the individual. In some embodiments, the oral care composition optionally further comprises fluoride and/or arginine.

In some embodiments of the methods of neutralizing toxicity of a lipopolysaccharide in an individual's oral cavity, the individual is identified as having inflammation of tissue their oral cavity. Some such individuals may be identified as having inflammation of tissue their oral cavity caused by a pro-inflammatory response stimulated by toxicity of a lipopolysaccharide in the oral cavity. Some such individuals may be identified as having plaque and inflammation in the oral cavity. In some embodiments, the individual is identified as having plaque and inflammation within an individual's gingival crevice. Individual treated with the methods may be identified as having plaque which comprises gram negative bacteria and inflammation in the oral cavity such as for example within an individual's gingival crevice.

### BRIEF DESCRIPTION OF THE DRAWINGS

FIG. 1 is an illustration of the cascade of inflammation mediated by NF-kB in a cell that occurs upon activation of TLR4 receptors by bacterial LPS. The cascade of inflammation results in secretion of proinflammatory cytokines such as for example IL-1β, which is depicted, and others such as IL-8, TNF-α, GM-CSF and IL-6 (not depicted).

FIG. 2 is an illustration depicting TLR4 activation of NF-kB.

FIG. 3 is bar graph showing concentrations of IL-8 in HEK-Blue cells supernatants with zinc oxide, zinc citrate, DZ and DZA solutions (600 ppm and 300 ppm for each) and standard P. gingivalis LPS (0.1 μg/ml).

FIG. 4 is bar graph showing concentrations of TNF-α in HEK-Blue cells supernatants with zinc oxide, zinc citrate, DZ and DZA solutions (600 ppm and 300 ppm for each) and standard P. gingivalis LPS (0.1 μg/ml).

FIG. 5 is bar graph showing concentrations of IL-8 in HEK-Blue cells supernatants with zinc oxide, zinc citrate, DZ and DZA solutions (10 mM and 1 mM for each) and standard P. gingivalis LPS (0.1 μg/ml).

FIG. 6 is bar graph showing viability of HEK-Blue cells co-incubated with various dilutions of zinc oxide, zinc citrate, DZ, DZA and arginine solutions.

FIG. 7 is bar graph showing IL-8 expression in HEK-Blue cells co-incubated with ultrapure P. gingivalis LPS (1 μg/ml) or various dilutions of zinc oxide, zinc citrate, DZ, DZA and arginine solutions.

FIG. 8 is bar graph showing concentrations of PGE₂ in human gingival 3D MatTek medium with slurries of control toothpaste or toothpaste containing DZA or DZA solution and E. coli LPS (10 μg/ml).

### DETAILED DESCRIPTION

Compositions which can interfere with the processes associated with LPS endotoxin activity and thereby prevent or reduce immune response stimulation by LPS can be useful components in oral care compositions such as toothpastes, oral rinses and mouth washes. Such compositions and methods are useful to reduce, eliminate or neutralize the endotoxic activity of LPS in the oral cavity. Such compositions are particularly useful in methods used prophylactically or when used to treat an individual who has been identified as having bacteria in their oral cavity that produces LPS endotoxin, particularly highly virulent LPS endotoxin. Compositions and methods for identifying compositions and methods that reduce, eliminate or neutralize the endotoxic activity of LPS in the oral cavity can be used in development and formulation of improved oral health products.

Methods for identifying a composition that neutralizes toxicity of a lipopolysaccharide are provided. The methods comprise performing a test assay, performing a control assay and comparing the results of each assay. The test assay comprises the contacting a Toll-like receptor reporter cell with an amount of a lipopolysaccharide sufficient to stimulate secretion of one or more proinflammatory cytokines and/or prostaglandin E2 by the Toll-like receptor reporter cell in the presence of a test composition, and measuring the amount of one or more proinflammatory cytokines and/or prostaglandin E2 secreted by the Toll receptor reporter cell in the test assay. The control assay comprises contacting, in the absence of the test composition, the Toll-like receptor reporter cell with the amount of the lipopolysaccharide sufficient to stimulate secretion of one or more proinflammatory cytokines and/or prostaglandin E2 by the Toll-like receptor reporter cell, and measuring the amount of one or more proinflammatory cytokines and/or prostaglandin E2 secreted by the Toll-like receptor reporter cell in the control assay. The amount of one or more proinflammatory cytokines and/or prostaglandin E2 secreted by the Toll-like receptor reporter cell in the test assay is compared with the amount of one or more proinflammatory cytokines and/or prostaglandin E2 secreted by the Toll-like receptor reporter cell in the control assay. If the amount of one or more proinflammatory cytokines and/or prostaglandin E2 secreted by the Toll-like receptor reporter cell in the test assay is less than the amount of one or more proinflammatory cytokines and/or prostaglandin E2 secreted by the Toll-like receptor reporter cell in the control assay, the test composition is identified as a composition that neutralizes toxicity of a lipopolysaccharide.

As used herein, a “Toll-like receptor reporter cell” and “TLR reporter cell” are used interchangeably and refer to a cell in culture which expresses one or more Toll-like receptors (TLR) that when activated by the endotoxin LPS stimulates the TLR reporter cell to secrete proinflammatory cytokines. Examples of TLRs expressed in a TLR reporter cell include TLR-2 and TLR-4. Other TLRs include TLR-1, TLR-3, TLR-5, TLR-6, TLR-7, TLR-8, TLR-9 and TLR-10.

The TLR reporter cell is typically a recombinant eukaryotic cell engineered to express one or more TLRs. In some embodiments, the TLR reporter cell is a eukaryotic cell selected from the group consisting of: a recombinant HEK 293T cell that expresses one or more TLRs, a recombinant human monocyte cell that expresses one or more TLRs, and a recombinant Chinese Hamster ovary cell that expresses one or more TLRs. In some embodiments, the TLR reporter cell is a eukaryotic cell selected from the group consisting of: a recombinant HEK 293T cell that expresses TLR2 and TLR4, a recombinant human monocyte cell that expresses TLR2 and TLR4, and a recombinant Chinese Hamster ovary cell that expresses TLR2 and TLR4. In some embodiments, the TLR reporter cell is a eukaryotic cell selected from the group consisting of: a recombinant HEK 293T cell that expresses TLR4, a recombinant human monocyte cell that expresses TLR-4, and a recombinant Chinese Hamster ovary cell that expresses TLR-4. In some embodiments, the TLR reporter cells is an HEK-Blue™ hTLR4 cells commercially available from Invitrogen.

In some embodiments, the human gingival tissue is used in assays and a human gingival cell of the human gingival tissue is the Toll-like receptor reporter cell. Human gingival cells of the human gingival tissue comprise one or more Toll-like receptors and may be recombinantly transformed to produce cells that express one or more additional Toll-like receptors. LPS induces human gingival cells of the human gingival tissue secrete prostaglandin. In some embodiments, the human gingival tissue is commercially available such as MatTek EpiGingival tissues, MatTek Corporation, cat #Gin-100.

The TLR reporter cell can be activated by an LPS and transduce a signal that induces secretion of one or more proinflammatory cytokines and/or prostaglandin E2.

Examples of proinflammatory cytokines secreted by TLR reporter cell include TNF-α, IL-6, IL-8, IL-1β and GM-CSF. In some embodiments, the amount of one or more proinflammatory cytokines secreted by the Toll-like receptor reporter cell is measured. In some embodiments, the amount of one or more proinflammatory cytokines secreted by the Toll-like receptor reporter cell is measured and the one or more proinflammatory cytokines is selected from the group consisting of: TNF-α, IL-6, IL-8, IL-1β and GM-CSF. In some embodiments, the amount of two or more proinflammatory cytokines secreted by the Toll-like receptor reporter cell are measured. In some embodiments, the amounts of two or more proinflammatory cytokines secreted by the Toll-like receptor reporter cell are measured and the two or more proinflammatory cytokines are selected from the group consisting of: TNF-α, IL-6, IL-8, IL-1β and GM-CSF. In some embodiments, the amounts of each of TNF-α, IL-6, IL-8, IL-1β and GM-CSF is measured. The amount of each proinflammatory cytokines secreted by the Toll-like receptor reporter cell may be measured using proinflammatory cytokine-specific magnetic beads such as TNF-α-specific magnetic beads, IL-6-specific magnetic beads, IL-8-specific magnetic beads, IL-1β-specific magnetic beads and GM-CSF-specific magnetic beads. In some embodiments, the amount of the IL-8 secreted by the Toll-like receptor reporter cell is measured. In some embodiments, the amount of the IL-8 secreted by the Toll-like receptor reporter cell is measured using IL-8-specific magnetic beads. In some embodiments, the amount of the TNF-α secreted by the Toll-like receptor reporter cell is measured. In some embodiments, the amount of the TNF-α, secreted by the Toll-like receptor reporter cell is measured using IL-8-specific magnetic beads. In some embodiments, the amount of the TNF-α, secreted by the Toll-like receptor reporter cell is measured using IL-8-specific magnetic beads.

In some embodiments, the amount of the prostaglandin E2 secreted by the Toll-like receptor reporter cell is measured using an ELISA assay such as the commercially available Enzo PGE 2 ELISA assay (Enzo Life Sciences, cat #ADI-900-001).

As used herein, “an amount of a lipopolysaccharide sufficient to stimulate secretion of one or more proinflammatory cytokines and/or prostaglandin E2 by the Toll-like receptor reporter cell” refers to an amount of a specific LPS used in a test or control assay that is recognized by and activates the TLR of the TLR reporter cell, inducing it secrete one or more proinflammatory cytokines and/or prostaglandin E2. The LPS used has endotoxin activity with respect to the TLR reporter cell used in the test and control assays. In the test and control assays, the amount of LPS used is determined by factors such as virulence. The virulence of LPS derived from different sources varies; the more virulent the LPS, the less is needed to stimulate secretion of one or more proinflammatory cytokine and/or prostaglandin E2 by the TLR reporter cell. The amount of LPS needed to stimulate detectable levels of one or proinflammatory cytokine and/or prostaglandin E2 secreted by the TLR reporter cell in a test or control assay can be routinely determined. The LPS may be isolated from bacterial sources and is typically provided in solution with an inactive solvent/diluent. LPS is typically characterized by its EC50. The LPS used is an LPS that activate the TLR expressed by the TLR reporter cell reporter cell and stimulates the TLR reporter cell to secrete proinflammatory cytokine and/or prostaglandin E2. LPS used is typically LPS derived from strains of pathogenic bacteria commonly found in the oral cavity, particularly in plaque and/or the gingival crevice. In some embodiments, the LPS used is LPS from Porphyromonas gingivalis (P. gingivalis), Escherichia coli (E. coli), Prevotella intermedia (P. intermedia), Fusobacterium nucleatum (F. nucleatum), Treponema denticola (T. denticola), Aggregatibacter actinomycetemcomitans (A. actinomycetemcomitans) or Tannerella forsythia (T. forsythia). Other pathogenic bacteria may be useful as a source such as for example, Eikenella corrodens, Campylobacter rectus, Campylobacter gracilis, Streptococcus mutans, Streptococcus sobrinus, Streptococcus sanguis, Streptococcus oralis, Actinomyces israelii, Chlamydia pneumoniae, Porphyromonas cangingivalis, Fusobacterium necrophorum, and Streptococcus constellatus. Purified LPS from P. gingivalis can be obtained from commercially available sources such as Sigma-Aldrich.

As used herein “a test composition” refers to the “active components” being assessed for the ability of neutralize LPS toxicity. The test composition may be present solely as active components or it may be part of a combination with inactive components. The test composition may be a single compound that is the active component or a combination of two or more compounds in which each of the two or more compounds are active components. In addition to the one or more active components, the test composition may be combined with inactive components. In some embodiments, the test composition is included in a mixture which contains active and inactive components. In some embodiments, the test composition is in a solution in which the solvent is an inactive component. In some embodiments, the test composition is one or more active components and is combined with saliva, which is an inactive component. In some embodiments, the test composition is the one or more active components that is combined with an oral care formulation such as a toothpaste formulation, a mouth wash formulation or an oral rinse formulation. Oral care formulations may include oral care ingredients as inactive components in combination one or more active components.

As used herein “the absence of a test composition” refers to the absence of the active component or components in a test composition but may include an inactive component or components. In particular, in the context of a control assay being performed with a test assay, the test assay may contact the TLR reporter cell with LPS and a test composition that is combined with inactive components and the control assay may contact the TLR reporter cell with LPS and the same inactive components used in the test assay but without the test composition.

In some embodiments, two or more test assays are performed using a series of concentrations of the test composition. In some embodiments, two or more test assays are performed using a series of concentrations of the lipopolysaccharide and two or more control assays are performed using the series of concentrations of the lipopolysaccharide.

Neutralizing endotoxic activity of a lipopolysaccharide refers to reduction in the effect of the lipopolysaccharide to stimulate an inflammatory response by a cell, particularly a cell in the oral cavity. Neutralizing LPS endotoxic activity may also be referred to as neutralizing LPS toxicity, detoxifying LPS, neutralizing toxicity or detoxifying and the like.

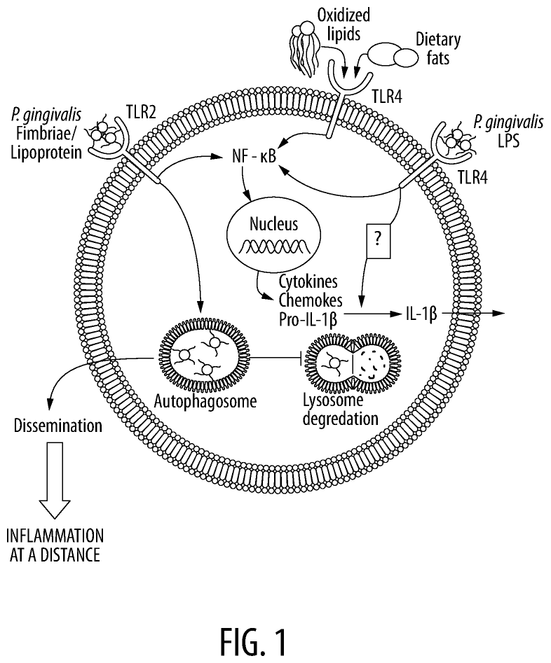

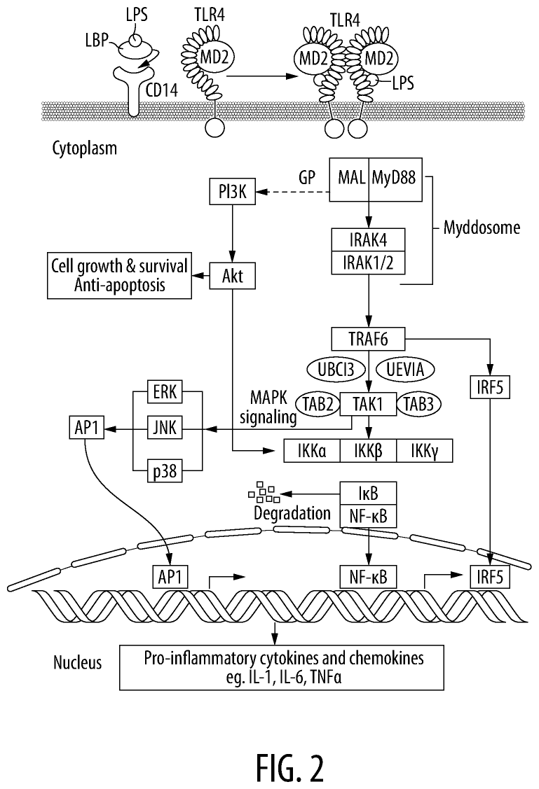

Without being bound by any specific theory, a test composition that neutralizes the endotoxic activity of a lipopolysaccharide may inhibit or reduce the activation of the TLR by the LPS by having an effect on the TLR, or by having an effect on the LPS, or by having an effect on the signal transduction that otherwise occurs following activation of the TLR or by having an effect on expression or secretion of the proinflammatory cytokine and/or prostaglandin E2 that would otherwise be caused by the signal transduction. Neutralizing endotoxic activity of a lipopolysaccharide may attenuate, reduce or inhibit the level of stimulation of an inflammatory response by a cell that occurs when the lipopolysaccharide activated the TLR, thereby reducing or inhibiting the amount or type of cytokines or other messengers and factors secreted. An inflammatory response by a cell may include activation of the NF-κB signaling pathway in the cell and/or a pro-inflammatory response such as the release of proinflammatory cytokines or other proinflammatory factors such as prostaglandin E2 by the cell and/or other activities engaged in an immune response. FIG. 1 illustrates activation of TLR4 by LPS stimulates an inflammatory response mediated by NF-κB. FIG. 2 illustrates TLR4 activation of NFκB. The endotoxic activity of an LPS is stimulation of a proinflammatory activity by a cell that is activated when the LPS binds to a receptor on the cell. Neutralizing the endotoxic activity refers to the inhibition, reduction, or elimination of such proinflammatory activity by cell in the presence of the LPS. Regardless of the mechanism, by neutralizing LPS endotoxic activity and detoxifying LPS, the LPS is less harmful and therefore the presence of the bacteria that produce it is less injurious.

In some embodiments, a method is provided to identify compositions that neutralize endotoxic activity of a lipopolysaccharide. In some embodiments, a model system is used which has been designed to allow quantitative monitoring of LPS endotoxicity. In the exemplary model system, TLR reporter cells HEK-Blue™ hTLR4 cells activated with P. gingivalis LPS secrete IL-8, which can be quantified. The model system can be used to identify compositions which modulate LPS endotoxicity. In addition, the model system can be used to compare relative toxicity levels of different LPS samples derived from different bacterial sources.

IL-8 may be quantified by well-known methods. In some embodiments, IL-8 is measured in cell culture supernatant using the commercially available Luminex Magpix instrument (MAGPIX-XPON42) and IL-8 specific magnetic beads such as those in the human 5-plex cytokine/chemokine Magnetic bead panel (Millpore HCYTOMAG-60K: TNF-α, IL-6, IL-8, IL-1β and GM-CSF). The commercially available Luminex Magpix instrument (MAGPIX-XPON42) is similarly useful to detect and quantify TNF-α, IL-6, IL-1β and GM-CSF).

In order to determine a test composition's efficacy to neutralize LPS endotoxic activity, an in vitro test assay may be performed in which a sample of TLR reporter cells such as HEK-Blue™ hTLR4 is contacted with a test sample of a lipopolysaccharide such as P. gingivalis LPS and a test sample of a test composition (with or without inactive components provided in combination with the test composition) after which the amount of IL-8 secreted by the TLR reporter cell is measured, such as by using a MAGPIX-XPON42 and a Millpore HCYTOMAG-60K Magnetic bead panel that includes IL-8 specific magnetic beads. The amount of IL-8 secreted by the TLR reporter cells in the test assay is compared to the amount of IL-8 secreted an identical sample of TLR reporter cells after being contacted with an identical sample of the lipopolysaccharide but in the absence of the test composition. If the test composition neutralizes LPS endotoxic activity, the amount of IL-8 secreted by the TLR reporter cell that is measured in the test assay is lower than the amount of IL-8 secreted by an identical TLR reporter cell contacted with an identical sample of LPS in the absence of the test composition. In some embodiments, amount of IL-8 secreted by an identical TLR reporter cell contacted with an identical sample of LPS in the absence of the test composition is a known standard based upon previously run control assays. In some embodiments, amount of IL-8 secreted by an identical TLR reporter cell contacted with an identical sample of LPS in the absence of the test composition is determined by running a negative control assay together with the test assay. The test assay and the negative control assay are identical in all respects except the test composition is absent. In embodiments, in which the test composition that includes one or more active components is combined with inactive components, the negative control assay may include a sample corresponding to the inactive components, such as solvent, saliva, and inactive formulation components.

Some embodiments may include a positive control assay which is identical to the test assay except instead of a sample of test composition, a sample of a positive control composition is used. A positive control composition contains an active component known to neutralize LPS endotoxin activity. The sample of positive control composition contains a known amount of known active component. Results from the positive control assay can thus be used to compare the neutralizing LPS endotoxin activity of the test composition relative to the neutralizing LPS endotoxin activity of the positive control composition. In some embodiments, the positive control composition comprises the known active component for neutralizing LPS endotoxin activity in a solution or mixture with inactive components. In some embodiments, the inactive components are identical to the inactive components used in the test composition.

The combination of zinc oxide and zinc citrate has been found to be particularly effective in neutralizing toxicity of LPS from P. gingivalis and thereby inhibit LPS-induced inflammation. The effectiveness of the combination of zinc oxide and zinc citrate exceeded the expected efficacy, showing efficacy greater than the additive effect of the active components when used individually.

The combination of zinc oxide and zinc citrate has been found to be particularly effective in neutralizing toxicity of LPS from P. gingivalis and thereby inhibit LPS-induced inflammation. The effectiveness of the combination of zinc oxide and zinc citrate exceeded the expected efficacy, showing efficacy greater than the additive effect of the active components when used individually.

Methods are provided for inhibiting inflammation within oral cavity an individual prophylactically. Methods are provided for inhibiting inflammation within oral cavity an individual who has been identified as having a pro-inflammatory response stimulated by toxicity of lipopolysaccharides in the oral cavity. Methods are provided for inhibiting inflammation within oral cavity an individual who has been identified as having gram-negative bacteria present which produce LPS that stimulates a pro-inflammatory response in the oral cavity. The methods comprise neutralizing lipopolysaccharide toxicity by applying an oral care composition to the individual's oral cavity in an amount effective to reduce lipopolysaccharide induced secretion of pro-inflammatory signals by cells in the oral cavity, wherein the oral care composition comprises a combination of zinc oxide and zinc citrate, and optionally may further comprise arginine and/or fluoride such as for example stannous fluoride. In some embodiments, the individual is identified as having a pro-inflammatory response stimulated by toxicity of lipopolysaccharides in the oral cavity by observing the presence of plaque and inflammation in the oral cavity. In some embodiments, the individual is identified as having a pro-inflammatory response stimulated by toxicity of lipopolysaccharides in the oral cavity by observing the presence of plaque and inflammation within an individual's gingival crevice. In some embodiments, the individual is identified as having a pro-inflammatory response stimulated by toxicity of lipopolysaccharides in the oral cavity by observing the presence of plaque which comprises gram negative bacteria and inflammation in the oral cavity. In some embodiments, the individual is identified as having a pro-inflammatory response stimulated by toxicity of lipopolysaccharides in the oral cavity by observing the presence of plaque which comprises gram negative bacteria and inflammation within an individual's gingival crevice. In some embodiments, the individual is identified as having a pro-inflammatory response stimulated by toxicity of lipopolysaccharides in the oral cavity by observing the presence of plaque which comprises P. gingivalis and inflammation in the oral cavity. In some embodiments, the individual is identified as having a pro-inflammatory response stimulated by toxicity of lipopolysaccharides in the oral cavity by observing the presence of plaque which comprises P. gingivalis and inflammation within an individual's gingival crevice. In some embodiments, the individual is identified as having a high potential of gum disease, such as gingivitis and periodontitis. Individuals may be identified by the presence of many anaerobic bacterial species, i.e. P. gingivalis, along the gum line and under the gum line.

Methods are provided for inhibiting inflammation within oral cavity an individual prophylactically. Methods are provided for preventing inflammation within oral cavity an individual. The methods comprise by applying an oral care composition to the individual's oral cavity in an amount effective to neutralizing lipopolysaccharide toxicity and reduce lipopolysaccharide induced secretion of pro-inflammatory signals by cells in the oral cavity, wherein the oral care composition comprises a combination of zinc oxide and zinc citrate, and optionally may further comprise arginine and/or fluoride such as for example stannous fluoride.

Embodiments provided herein include methods of neutralizing LPS toxicity and preventing or inhibiting inflammation that comprise applying to the oral cavity of an individual an oral care composition that comprises an effective amount of zinc oxide and zinc citrate, and optionally may further comprise arginine and/or fluoride such as for example stannous fluoride. In some embodiments, oral care compositions are a toothpaste or a mouthwash.

In some embodiments the oral care compositions comprise zinc oxide to zinc citrate in a ratio from 1.5:1 to 4.5:1, 1.5:1 to 4:1, 1.7:1 to 2.3:1, 1.9:1 to 2.1:1, or about 2:1. Also, the corresponding molar ratios based on these weight ratios can be used. In some embodiments, the total concentration of zinc salts in the composition is from 0.2 weight % to 5 weight %, or from 0.5 weight % to 2.5 weight % or from 0.8 weight % to 2 weight %, or about 1.5 weight %, based on the total weight of the composition. In some embodiments, the molar ratio of arginine to total zinc salts is from 0.05:1 to 10:1. In some embodiments, the composition comprises zinc oxide in an amount of from 0.5 weight % to 1.5 weight % and zinc citrate in an amount of from 0.25 weight % to 0.75 weight %, based on the total weight of the composition. In some embodiments, the composition may comprise zinc oxide in an amount of from 0.75 weight % to 1.25 weigh % and zinc citrate in an amount of from 0.4 weight % to 0.6 weight %, based on the total weight of the composition. In some embodiments, the composition comprises zinc oxide in an amount of about 1 weight % and zinc citrate in an amount of about 0.5 weight %, based on the total weight of the composition. In some embodiments, zinc oxide may be present in an amount of from 0.75 to 1.25 wt % (e.g., 1.0 wt. %) the zinc citrate is in an amount of from 0.25 to 1.0 wt % (e.g. 0.25 to 0.75 wt. %, or 0.5 wt. %) and based on the weight of the oral care composition. In some embodiments, the zinc citrate is about 0.5 wt %. In some embodiments, the zinc oxide is about 1.0 wt %.

In some embodiments the ZnO particles may have an average particle size of from 1 to 7 microns. In some embodiments, the ZnO particles have an average particle size of 5 microns or less. In some embodiments, suitable zinc oxide particles for oral care compositions have, for example, a particle size distribution of 3 to 4 microns, or alternatively, a particle size distribution of 5 to 7 microns, alternatively, a particle size distribution of 3 to 5 microns, alternatively, a particle size distribution of 2 to 5 microns, or alternatively, a particle size distribution of 2 to 4 microns. Zinc oxide may have a particle size which is a median particle size. Suitable particles may have, for example, a median particle size of 8 microns or less, alternatively, a median particle size of 3 to 4 microns, alternatively, a median particle size of 5 to 7 microns, alternatively, a median particle size of 3 to 5 microns, alternatively, a median particle size of 2 to 5 microns, or alternatively, a median particle size of 2 to 4 microns. In another aspect, that particle size is an average (mean) particle size. In an embodiment, the mean particle comprises at least 5%, at least 10%, at least 15%, at least 20%, at least 25%, at least 30%, at least 35%, or at least 40% of the total metal oxide particles in an oral care composition of the invention. The particle may be present in an amount of up to 5% by weight, based on the total weight of the oral care composition, for example in an amount of from 0.5 to 5% by weight, preferably of up to 2% by weight, more preferably from 0.5 to 2% by weight, more preferably from 1 to 2% by weight, or in some embodiment from 2.5 to 4.5% by weight, being based on the total weight of the oral care composition. In some embodiments, the source of zinc oxide particles and/or the form they may be incorporated into the oral care composition in is selected from one or more of a powder, a nanoparticle solution or suspension, or encapsulated in a polymer or bead. Zinc oxide particles may be selected to achieve occlusion of dentin particles. Particle size distribution may be measured using a Malvern Particle Size Analyzer, Model Mastersizer 2000 (or comparable model) (Malvern Instruments, Inc., Southborough, Mass.), wherein a helium-neon gas laser beam is projected through a transparent cell which contains silica, such as, for example, silica hydrogel particles suspended in an aqueous solution. Light rays which strike the particles are scattered through angles which are inversely proportional to the particle size. The photodetector arrant measures the quantity of light at several predetermined angles. Electrical signals proportional to the measured light flux values are then processed by a microcomputer system, against a scatter pattern predicted from theoretical particles as defined by the refractive indices of the sample and aqueous dispersant to determine the particle size distribution of the metal oxide. It will be understood that other methods of measuring particle size are known in the art, and based on the disclosure set forth herein, the skilled artisan will understand how to calculate median particle size, mean particle size, and/or particle size distribution of metal oxide particles.

Oral care compositions optionally comprise clinically effective form of fluoride. Stannous fluoride may be present in a clinically efficacious amount. Fluoride where present may be present at levels of, e.g., about 25 to about 25,000 ppm, for example about 50 to about 5000 ppm, about 750 to about 2,000 ppm for a consumer toothpaste (e.g., 1000-1500 ppm, e.g., about 1000 ppm, e.g., about 1450 ppm). In some embodiments, fluoride is present from about 100 to about 1000, from about 200 to about 500, or about 250 ppm fluoride ion. 500 to 3000 ppm. In some embodiments, the fluoride source provides fluoride ion in an amount of from 50 to 25,000 ppm (e.g., 750-7000 ppm, e.g., 1000-5500 ppm, e.g., about 500 ppm, 1000 ppm, 1100 ppm, 2800 ppm, 5000 ppm, or 25000 ppm). In some embodiments, the fluoride source is stannous fluoride which provides fluoride in an amount from 750-7000 ppm (e.g., about 1000 ppm, 1100 ppm, 2800 ppm, 5000 ppm). In some embodiments, the fluoride source is stannous fluoride which provides fluoride in an amount of about 5000 ppm. Fluoride ion sources may be added to the compositions at a level of about 0.001 wt. % to about 10 wt. %, e.g., from about 0.003 wt. % to about 5 wt. %, 0.01 wt. % to about 1 wt., or about 0.05 wt. %. In some embodiment, the stannous fluoride is present in an amount of 0.1 wt. % to 2 wt. % (0.1 wt %-0.6 wt. %) of the total composition weight.

Oral care compositions optionally comprise arginine or a salt thereof. In some embodiments, the arginine is L-arginine or a salt thereof. Suitable salts include salts known in the art to be pharmaceutically acceptable salts are generally considered to be physiologically acceptable in the amounts and concentrations provided. Physiologically acceptable salts include those derived from pharmaceutically acceptable inorganic or organic acids or bases, for example acid addition salts formed by acids which form a physiological acceptable anion, e.g., hydrochloride or bromide salt, and base addition salts formed by bases which form a physiologically acceptable cation, for example those derived from alkali metals such as potassium and sodium or alkaline earth metals such as calcium and magnesium. Physiologically acceptable salts may be obtained using standard procedures known in the art, for example, by reacting a sufficiently basic compound such as an amine with a suitable acid affording a physiologically acceptable anion. In some embodiments, the arginine in partially or wholly in salt form such as arginine phosphate, arginine hydrochloride or arginine bicarbonate. In some embodiments, the arginine is present in an amount corresponding to 0.1% to 15%, e.g., 0.1 wt % to 10 wt %, e.g., 0.1 to 5 wt %, e.g., 0.5 wt % to 3 wt % of the total composition weight, about e.g., 1%, 1.5%, 2%, 3%, 4%, 5%, or 8%, wherein the weight of the arginine is calculated as free form. In some embodiments the arginine is present in an amount corresponding to about 0.5 wt. % to about 20 wt. % of the total composition weight, about 0.5 wt. % to about 10 wt. % of the total composition weight, for example about 1.5 wt. %, about 3.75 wt. %, about 5 wt. %, or about 7.5 wt. % wherein the weight of the arginine is calculated as free form. In some embodiments, the arginine is present in an amount of from 0.5 weight % to 10 weight %, or from 0.5 weight % to 3 weight % or from 1 weight % to 2.85 weight %, or from 1.17 weight % to 2.25 weight %, based or from 1.4 weight % to 1.6 weight %, or from 0.75 weight % to 2.9 weight %, or from 1.3 weight % to 2 weight %, or about 1.5 weight %, based on the total weight of the composition. Typically, the arginine is present in an amount of up to 5% by weight, further optionally from 0.5 to 5% by weight, still further optionally from 2.5 to 4.5% by weight, based on the total weight of the oral care composition. In some embodiments, arginine is present in an amount from 0.1 wt. %-6.0 wt. %. (e.g., about 1.5 wt %) or from about 4.5 wt. %-8.5 wt. % (e.g., 5.0%) or from 3.5 wt. %-9 wt. % or 8.0 wt. %. In some embodiments, the arginine is present in a dentifrice, at for example about 0.5-2 wt. %, e.g., and about 0.8% in the case of a mouthwash.

The oral care compositions optionally comprise zingerone. In some embodiments, the zingerone is present in an amount of from 0.01% to 1% (e.g., 0.05% to 0.5%; e.g., 0.05% to 0.35%; e.g., 0.1%, 0.2%, or 0.3%),

The oral care compositions described herein may also comprise one or more further agents such as those typically selected from the group consisting of: abrasives, an anti-plaque agent, a whitening agent, antibacterial agent, cleaning agent, a flavoring agent, a sweetening agent, adhesion agents, surfactants, foam modulators, pH modifying agents, humectants, mouth-feel agents, colorants, tartar control (anti-calculus) agent, polymers, saliva stimulating agent, nutrient, viscosity modifier, anti-sensitivity agent, antioxidant, and combinations thereof.

In some embodiments, the oral care compositions comprise one or more abrasive particulates such as those useful for example as a polishing agent. Any orally acceptable abrasive can be used, but type, fineness, (particle size) and amount of abrasive should be selected so that tooth enamel is not excessively abraded in normal use of the composition. Examples of abrasive particulates may be used include abrasives such sodium bicarbonate, insoluble phosphates (such as orthophosphates, polymetaphosphates and pyrophosphates including dicalcium orthophosphate dihydrate, calcium pyrophosphate, tricalcium phosphate, calcium polymetaphosphate and insoluble sodium polymetaphosphate), calcium phosphate (e.g., dicalcium phosphate dihydrate), calcium sulfate, natural calcium carbonate (CC), precipitated calcium carbonate (PCC), silica (e.g., hydrated silica or silica gels or in the form of precipitated silica or as admixed with alumina), iron oxide, aluminum oxide, aluminum silicate, calcined alumina, bentonite, other siliceous materials, perlite, plastic particles, e.g., polyethylene, and combinations thereof. The natural calcium carbonate abrasive of is typically a finely ground limestone which may optionally be refined or partially refined to remove impurities. The material preferably has an average particle size of less than 10 microns, e.g., 3-7 microns, e.g. about 5.5 microns. For example, a small particle silica may have an average particle size (D50) of 2.5-4.5 microns. Because natural calcium carbonate may contain a high proportion of relatively large particles of not carefully controlled, which may unacceptably increase the abrasivity, preferably no more than 0.01%, preferably no more than 0.004%) by weight of particles would not pass through a 325 mesh. The material has strong crystal structure, and is thus much harder and more abrasive than precipitated calcium carbonate. The tap density for the natural calcium carbonate is for example between 1 and 1.5 g/cc, e.g., about 1.2 for example about 1.19 g/cc. There are different polymorphs of natural calcium carbonate, e.g., calcite, aragonite and vaterite, calcite being preferred for purposes of this invention. An example of a commercially available product suitable for use in the present invention includes Vicron® 25-11 FG from GMZ. Precipitated calcium carbonate has a different crystal structure from natural calcium carbonate. It is generally more friable and more porous, thus having lower abrasivity and higher water absorption. For use in the present invention, the particles are small, e.g., having an average particle size of 1-5 microns, and e.g., no more than 0.1%, preferably no more than 0.05% by weight of particles which would not pass through a 325 mesh. The particles may for example have a D50 of 3-6 microns, for example 3.8-4.9, e.g., about 4.3; a D50 of 1-4 microns, e.g. 2.2-2.6 microns, e.g., about 2.4 microns, and a D10 of 1-2 microns, e.g., 1.2-1.4, e.g. about 1.3 microns. The particles have relatively high-water absorption, e.g., at least 25 g/100 g, e.g. 30-70 g/100 g. Examples of commercially available products suitable for use include, for example, Carbolag® 15 Plus from Lagos Industria Quimica. In some embodiments, additional calcium-containing abrasives, for example calcium phosphate abrasive, e.g., tricalcium phosphate, hydroxyapatite or dicalcium phosphate dihydrate or calcium pyrophosphate, and/or silica abrasives, sodium metaphosphate, potassium metaphosphate, aluminum silicate, calcined alumina, bentonite or other siliceous materials, or combinations thereof are used. Examples of silica abrasives include, but are not limited to, precipitated or hydrated silicas having a mean particle size of up to about 20 microns (such as Zeodent 105 and Zeodent 1 14 marketed by J. M. Huber Chemicals Division, Havre de Grace, Md. 21078); Sylodent 783 (marketed by Davison Chemical Division of W.R. Grace & Company); or Sorbosil AC 43 (from PQ Corporation). In some embodiments, an effective amount of a silica abrasive is about 10-30%, e.g. about 20%. In some embodiments, the acidic silica abrasive Sylodent is included at a concentration of about 2 to about 35% by weight; about 3 to about 20% by weight, about 3 to about 15% by weight, about 10 to about 15% by weight. For example, the acidic silica abrasive may be present in an amount selected from 2 wt. %, 3 wt. %, 4% wt. %, 5 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %. Sylodent 783 has a pH of 3.4-4.2 when measured as a 5% by weight slurry in water and silica material has an average particle size of less than 10 microns, e.g., 3-7 microns, e.g. about 5.5 microns. In some embodiments, the silica is synthetic amorphous silica, (e.g., 1%-28% by wt.) (e.g., 8%-25% by wt). In some embodiments, the silica abrasives are silica gels or precipitated amorphous silicas, e.g. silicas having an average particle size ranging from 2.5 microns to 12 microns. Some embodiments further comprise a small particle silica having a median particle size (d50) of 1-5 microns (e.g., 3-4 microns) (e.g., about 5 wt. % Sorbosil AC43 from PQ Corporation Warrington, United Kingdom). The composition may contain from 5 to 20 wt % small particle silica, or for example 10-15 wt %, or for example 5 wt %, 10 wt %, 15 wt % or 20 wt % small particle silica. In some embodiments, 20-30 wt % of the total silica in the composition is small particle silica (e.g., having a median particle size (d50) of 3-4 microns and wherein the small particle silica is about 5 wt. % of the oral care composition. In some embodiments, silica is used as a thickening agent, e.g., particle silica. In some embodiments, the composition comprises calcium carbonate, such as precipitated calcium carbonate high absorption (e.g., 20% to 30% by weight of the composition or, 25% precipitated calcium carbonate high absorption), or precipitated calcium carbonate-light (e.g., about 10% precipitated calcium carbonate-light) or about 10% natural calcium carbonate.

In some embodiments, the oral care compositions comprise a whitening agent, e.g., a selected from the group consisting of peroxides, metal chlorites, perborates, percarbonates, peroxyacids, hypochlorites, hydroxyapatite, and combinations thereof. Oral care compositions may comprise hydrogen peroxide or a hydrogen peroxide source, e.g., urea peroxide or a peroxide salt or complex (e.g., such as peroxyphosphate, peroxycarbonate, perborate, peroxysilicate, or persulphate salts; for example, calcium peroxyphosphate, sodium perborate, sodium carbonate peroxide, sodium peroxyphosphate, and potassium persulfate or hydrogen peroxide polymer complexes such as hydrogen peroxide-polyvinyl pyrrolidone polymer complexes.

In some embodiments, the oral care compositions comprise an effective amount of one or more antibacterial agents, for example comprising an antibacterial agent selected from halogenated diphenyl ether (e.g. triclosan), triclosan monophosphate, herbal extracts and essential oils (e.g., rosemary extract, tea extract, magnolia extract, thymol, menthol, eucalyptol, geraniol, carvacrol, citral, hinokitol, magonol, ursolic acid, ursic acid, morin, catechol, methyl salicylate, epigallocatechin gallate, epigallocatechin, gallic acid, miswak extract, sea-buckthorn extract), bisguanide antiseptics (e.g., chlorhexidine, alexidine or octenidine), quaternary ammonium compounds (e.g., cetylpyridinium chloride (CPC), benzalkonium chloride, tetradecylpyridinium chloride (TPC), N-tetradecyl-4-ethylpyridinium chloride (TDEPC)), phenolic antiseptics, hexetidine furanones, bacteriocins, ethyllauroyl arginate, arginine bicarbonate, a Camellia extract, a flavonoid, a flavan, halogenated diphenyl ether, creatine, sanguinarine, povidone iodine, delmopinol, salifluor, metal ions (e.g., zinc salts, stannous salts, copper salts, iron salts), propolis and oxygenating agents (e.g., hydrogen peroxide, buffered sodium peroxyborate or peroxycarbonate), phthalic acid and its salts, monoperthalic acid and its salts and esters, ascorbyl stearate, oleoyl sarcosine, alkyl sulfate, dioctyl sulfosuccinate, salicylanilide, domiphen bromide, delmopinol, octapinol and other piperidino derivatives, nisin preparations, chlorite salts; parabens such as methylparaben or propylparaben and mixtures of any of the foregoing. One or more additional antibacterial or preservative agents may optionally be present in the composition in a total amount of from about 0.01 wt. % to about 0.5 wt. %, optionally about 0.05 wt. % to about 0.1 wt. % or about 0.3%. by total weight of the composition.

In some embodiments, the oral care compositions may comprise at least one bicarbonate salt useful for example to impart a “clean feel” to teeth and gums due to effervescence and release of carbon dioxide. Any orally acceptable bicarbonate can be used, including without limitation, alkali metal bicarbonates such as sodium and potassium bicarbonates, ammonium bicarbonate and the like. The one or more additional bicarbonate salts are optionally present in a total amount of about 0.1 wt. % to about 50 wt. %, for example about 1 wt. % to 20 wt. %, by total weight of the composition.

In some embodiments, the oral care compositions also comprise at least one flavorant, useful for example to enhance taste of the composition. Any orally acceptable natural or synthetic flavorant can be used, including without limitation essential oils and various flavoring aldehydes, esters, alcohols, and similar materials, tea flavors, vanillin, sage, marjoram, parsley oil, spearmint oil, cinnamon oil, oil of wintergreen, peppermint oil, clove oil, bay oil, anise oil, eucalyptus oil, citrus oils, fruit oils, sassafras and essences including those derived from lemon, orange, lime, grapefruit, apricot, banana, grape, apple, strawberry, cherry, pineapple, etc., bean- and nut-derived flavors such as coffee, cocoa, cola, peanut, almond, etc., adsorbed and encapsulated flavorants and the like. Also encompassed within flavorants herein are ingredients that provide fragrance and/or other sensory effect in the mouth, including cooling or warming effects. Such ingredients illustratively include menthol, carvone, menthyl acetate, menthyl lactate, camphor, eucalyptus oil, eucalyptol, anethole, eugenol, cassia, oxanone, a-irisone, propenyl guaiethoi, thymol, linalool, benzaldehyde, cinnamaldehyde, N-ethyl-p-menthan-3-carboxamine, N,2,3-trimethyl-2-isopropylbutanamide, 3-(1-menthoxy)-propane-1,2-diol, cinnamaldehyde glycerol acetal (CGA), menthone glycerol acetal (MGA) and the like. One or more flavorants are optionally present in a total amount of from about 0.01 wt. % to about 5 wt. %, for example, from about 0.03 wt. % to about 2.5 wt. %, optionally about 0.05 wt. % to about 1.5 wt. %, further optionally about 0.1 wt. % to about 0.3 wt. % and in some embodiments in various embodiments from about 0.01 wt. % to about 1 wt. %, from about 0.05 to about 2%, from about 0.1% to about 2.5%, and from about 0.1 to about 0.5% by total weight of the composition.

In some embodiments, the oral care compositions comprise at least one sweetener, useful for example to enhance taste of the composition. Sweetening agents among those useful herein include dextrose, polydextrose, sucrose, maltose, dextrin, dried invert sugar, mannose, xylose, ribose, fructose, levulose, galactose, corn syrup, partially hydrolyzed starch, hydrogenated starch hydrolysate, ethanol, sorbitol, mannitol, xylitol, maltitol, isomalt, aspartame, neotame, saccharin and salts thereof (e.g. sodium saccharin), sucralose, dipeptide-based intense sweeteners, cyclamates, dihydrochalcones, glycerine, propylene glycol, polyethylene glycols, Poloxomer polymers such as POLOXOMER 407, PLURONIC F108, (both available from BASF Corporation), alkyl polyglycoside (APG), polysorbate, PEG40, castor oil, menthol, and mixtures thereof. One or more sweeteners are optionally present in a total amount depending strongly on the particular sweetener(s) selected, but typically 0.005 wt. % to 5 wt. %, by total weight of the composition, optionally 0.005 wt. % to 0.2 wt. %, further optionally 0.05 wt. % to 0.1 wt. % by total weight of the composition.

In some embodiments, the oral care compositions further comprise an agent that interferes with or prevents bacterial attachment, e.g., ethyl lauroyl arginate (ELA), solbrol or chitosan, as well as plaque dispersing agents such as enzymes (papain, glucoamylase, etc.).

In some embodiments, the oral care compositions also comprise at least one surfactant. Any orally acceptable surfactant, most of which are anionic, cationic, zwitterionic, nonionic or amphoteric, and mixtures thereof, can be used. Examples of suitable surfactants include water-soluble salts of higher fatty acid monoglyceride monosulfates, such as the sodium salt of monosulfated monoglyceride of hydrogenated coconut oil fatty acids; higher alkyl sulfates such as sodium lauryl sulfate, sodium coconut monoglyceride sulfonate, sodium lauryl sarcosinate, sodium lauryl isoethionate, sodium laureth carboxylate and sodium dodecyl benzenesulfonate; alkyl aryl sulfonates such as sodium dodecyl benzene sulfonate; higher alkyl sulfoacetates, such as sodium lauryl sulfoacetate; higher fatty acid esters of 1,2-dihydroxypropane sulfonate; and the substantially saturated higher aliphatic acyl amides of lower aliphatic amino carboxylic compounds, such as those having 12-16 carbons in the fatty acid, alkyl or acyl radicals; and the like. Examples of amides include N-lauryl sarcosine, and the sodium, potassium and ethanolamine salts of N-lauryl, N-myristoyl, or N-palmitoyl sarcosine. Examples of cationic surfactants include derivatives of aliphatic quaternary ammonium compounds having one long alkyl chain containing 8 to 18 carbon atoms such as lauryl trimethylammonium chloride, cetyl pyridinium chloride, cetyl trimethyl ammonium bromide, di-isobutylphenoxyethyldimethylbenzylammonium chloride, coconut alkyltrimethylammonium nitrite, cetyl pyridinium fluoride, and mixtures thereof. Suitable nonionic surfactants include without limitation, poloxamers, polyoxyethylene sorbitan esters, fatty alcohol ethoxylates, alkylphenol ethoxylates, tertiary amine oxides, tertiary phosphine oxides, di alkyl sulfoxides and the like. Others include, for example, non-anionic polyoxyethylene surfactants, such as Polyoxamer 407, Steareth 30, Polysorbate 20, and castor oil; and amphoteric surfactants such as derivatives of aliphatic secondary and tertiary amines having an anionic group such as carboxylate, sulfate, sulfonate, phosphate or phosphonate such as cocamidopropyl betaine (tegobaine), and cocamidopropyl betaine lauryl glucoside; condensation products of ethylene oxide with various hydrogen containing compounds that are reactive therewith and have long hydrocarbon chains (e.g., aliphatic chains of from 12 to 20 carbon atoms), which condensation products (ethoxamers) contain hydrophilic polyoxyethylene moieties, such as condensation products of poly (ethylene oxide) with fatty acids, fatty, alcohols, fatty amides and other fatty moieties, and with propylene oxide and polypropylene oxides. In some embodiments, the oral composition includes a surfactant system that is sodium laurel sulfate (SLS) and cocamidopropyl betaine. One or more surfactants are optionally present in a total amount of about 0.01 wt. % to about 10 wt. %, for example, from about 0.05 wt. % to about 5 wt. %, or from about 0.1 wt. % to about 2 wt. %, e.g 1.5% wt. by total weight of the composition. In some embodiments, the oral composition include an anionic surfactant, e.g., a surfactant selected from sodium lauryl sulfate, sodium ether lauryl sulfate, and mixtures thereof, e.g. in an amount of from about 0.3% to about 4.5% by weight, e.g. 1-2% sodium lauryl sulfate (SLS); and/or a zwitterionic surfactant, for example a betaine surfactant, for example cocamidopropylbetaine, e.g. in an amount of from about 0.1% to about 4.5% by weight, e.g. 0.5-2% cocamidopropylbetaine. Some embodiments comprise a nonionic surfactant in an amount of from 0.5-5%, e.g, 1-2%, selected from poloxamers (e.g., poloxamer 407), polysorbates (e.g., polysorbate 20), polyoxyl hydrogenated castor oil (e.g., polyoxyl 40 hydrogenated castor oil), and mixtures thereof. In some embodiments, the poloxamer nonionic surfactant has a polyoxypropylene molecular mass of from 3000 to 5000 g/mol and a polyoxyethylene content of from 60 to 80 mol %, e.g., the poloxamer nonionic surfactant comprises poloxamer 407. Any of the preceding compositions may further comprise sorbitol, wherein the sorbitol is in a total amount of 10-40% (e.g., about 23%).

In some embodiments, the oral care compositions comprise at least, one foam modulator, useful for example to increase amount, thickness or stability of foam generated by the composition upon agitation. Any orally acceptable foam modulator can be used, including without limitation, polyethylene glycols (PEGs), also known as polyoxyethylenes. High molecular weight PEGs are suitable, including those having an average molecular weight of 200,000 to 7,000,000, for example 500,000 to 5,000,000, or 1,000,000 to 2,500,000, One or more PEGs are optionally present in a total amount of about 0.1 wt. % to about 10 wt. %, for example from about 0.2 wt. % to about 5 wt. %, or from about 0.25 wt. % to about 2 wt. %, by total weight of the composition

In some embodiments, the oral care compositions comprise at least one pH modifying agent. Such agents include acidifying agents to lower pH, basifying agents to raise pH, and buffering agents to control pH within a desired range. For example, one or more compounds selected from acidifying, basifying and buffering agents can be included to provide a pH of 2 to 10, or in various illustrative embodiments, 2 to 8, 3 to 9, 4 to 8, 5 to 7, 6 to 10, 7 to 9, etc. Any orally acceptable pH modifying agent can be used, including without limitation, carboxylic, phosphoric and sulfonic acids, acid salts (e.g., monosodium citrate, disodium citrate, monosodium malate, etc.), alkali metal hydroxides such as sodium hydroxide, carbonates such as sodium carbonate, bicarbonates such as sodium bicarbonate, sesquicarbonates, borates, silicates, bisulfates, phosphates (e.g., monosodium phosphate, trisodium phosphate, monopotassium phosphate, dipotassium phosphate, tribasic sodium phosphate, sodium tripolyphosphate, phosphoric acid), imidazole, sodium phosphate buffer (e.g., sodium phosphate monobasic and disodium phosphate) citrates (e.g. citric acid, trisodium citrate dehydrate), pyrophosphates (sodium and potassium salts) and the like and combinations thereof. One or more pH modifying agents are optionally present in a total amount effective to maintain the composition in an orally acceptable pH range. Compositions may have a pH that is either acidic or basic, e.g., from pH 4 to pH 5.5 or from pH 8 to pH 10. In some embodiments, the amount of buffering agent is sufficient to provide a pH of about 5 to about 9, preferable about 6 to about 8, and more preferable about 7, when the composition is dissolved in water, a mouthrinse base, or a toothpaste base. Typical amounts of buffering agent are about 5% to about 35%, in one embodiment about 10% to about 30%), in another embodiment about 15% to about 25%, by weight of the total composition.

In some embodiments, the oral care compositions also comprise at least one humectant. Any orally acceptable humectant can be used, including without limitation, polyhydric alcohols such as glycerin, sorbitol (optionally as a 70 wt. % solution in water), propylene glycol, xylitol or low molecular weight polyethylene glycols (PEGs) and mixtures thereof. Most humectants also function as sweeteners. In some embodiments, compositions comprise 15% to 70% or 30% to 65% by weight humectant. Suitable humectants include edible polyhydric alcohols such as glycerine, sorbitol, xylitol, propylene glycol as well as other polyols and mixtures of these humectants. Mixtures of glycerine and sorbitol may be used in certain embodiments as the humectant component of the compositions herein. One or more humectants are optionally present in a total amount of from about 1 wt. % to about 70 wt. %, for example, from about 1 wt. % to about 50 wt. %, from about 2 wt. % to about 25 wt. %, or from about 5 wt. % to about 15 wt. %, by total weight of the composition. In some embodiments, humectants, such as glycerin are present in an amount that is at least 20%>, e.g., 20-40%, e.g., 25-35%.

Mouth-feel agents include materials imparting a desirable texture or other feeling during use of the composition. In some embodiments, the oral care compositions comprise at least one thickening agent, useful for example to impart a desired consistency and/or mouth feel to the composition. Any orally acceptable thickening agent can be used, including without limitation, carbomers, also known as carboxyvinyl polymers, carrageenans, also known as Irish moss and more particularly i-carrageenan (iota-carrageenan), cellulosic polymers such as hydroxyethyl cellulose, and water-soluble salts of cellulose ethers (e.g., sodium carboxymethyl cellulose and sodium carboxymethyl hydroxyethyl cellulose), carboxymethylcellulose (CMC) and salts thereof, e.g., CMC sodium, natural gums such as karaya, xanthan, gum arabic and tragacanthin, colloidal magnesium aluminum silicate, colloidal silica, starch, polyvinyl pyrrolidone, hydroxyethyl propyl cellulose, hydroxybutyl methyl cellulose, hydroxypropyl methyl cellulose, and hydroxyethyl cellulose and amorphous silicas, and the like. A preferred class of thickening or gelling agents includes a class of homopolymers of acrylic acid crosslinked with an alkyl ether of pentaerythritol or an alkyl ether of sucrose, or carbomers. Carbomers are commercially available from B. F. Goodrich as the Carbopol© series. Particularly preferred Carbopols include Carbopol 934, 940, 941, 956, 974P, and mixtures thereof. Silica thickeners such as DT 267 (from PPG Industries) may also be used. One or more thickening agents are optionally present in a total amount of from about 0.01 wt. % to 15 wt. %, for example from about 0.1 wt. % to about 10 wt. %, or from about 0.2 wt. % to about 5 wt. %, by total weight of the composition. Some embodiments comprise sodium carboxymethyl cellulose (e.g., from 0.5 wt. %-1.5 wt. %). In certain embodiments, thickening agents in an amount of about 0.5% to about 5.0% by weight of the total composition are used. Thickeners may be present in an amount of from 1 wt % to 15 wt %, from 3 wt % to 10 wt %, 4 wt % to 9 wt %, from 5 wt % to 8 wt %, for example 5 wt %, 6 wt %, 7 wt %, or 8 wt %.

In some embodiments, the oral care compositions comprise at least one colorant. Colorants herein include pigments, dyes, lakes and agents imparting a particular luster or reflectivity such as pearling agents. In various embodiments, colorants are operable to provide a white or light-colored coating on a dental surface, to act as an indicator of locations on a dental surface that have been effectively contacted by the composition, and/or to modify appearance, in particular color and/or opacity, of the composition to enhance attractiveness to the consumer. Any orally acceptable colorant can be used, including FD&C dyes and pigments, talc, mica, magnesium carbonate, calcium carbonate, magnesium silicate, magnesium aluminum silicate, silica, titanium dioxide, zinc oxide, red, yellow, brown and black iron oxides, ferric ammonium ferrocyanide, manganese violet, ultramarine, titaniated mica, bismuth oxychloride, and mixtures thereof. One or more colorants are optionally present in a total amount of about 0.001% to about 20%, for example about 0.01% to about 10% or about 0.1% to about 5% by total weight of the composition.

In some embodiments, the oral care composition further comprises an anti-calculus (tartar control) agent. Suitable anti-calculus agents include, but are not limited to: phosphates and polyphosphates, polyaminopropane sulfonic acid (AM PS), polyolefin sulfonates, polyolefin phosphates, diphosphonates such as azacycloalkane-2,2-diphosphonates (e.g., azacycloheptane-2,2-diphosphonic acid), N-methyl azacyclopentane-2,3-diphosphonic acid, ethane-1-hydroxy-1,1-diphosphonic acid (EHIDP) and ethane-1-amino-1,1-diphosphonate, phosphonoalkane carboxylic acids and. Useful inorganic phosphate and polyphosphate salts include monobasic, dibasic and tribasic sodium phosphates. Soluble pyrophosphates are useful anticalculus agents. The pyrophosphate salts can be any of the alkali metal pyrophosphate salts. In certain embodiments, salts include tetra alkali metal pyrophosphate, dialkali metal diacid pyrophosphate, trialkali metal monoacid pyrophosphate and mixtures thereof, wherein the alkali metals are sodium or potassium. The pyrophosphates also contribute to preservation of the compositions by lowering water activity, tetrasodium pyrophosphate (TSPP), tetrapotassium pyrophosphate, sodium tripolyphosphate, tetrapolyphosphate, sodium trimetaphosphate, sodium hexametaphosphate and mixtures thereof. The salts are useful in both their hydrated and unhydrated forms. An effective amount of pyrophosphate salt useful in the present composition is generally enough to provide least 0.1 wt. % pyrophosphate ions, e.g., 0.1 to 3 wt. %, e.g., 0.1 to 2 wt. %, e.g., 0.1 to 1 wt. %, e.g., 0.2 to 0.5 wt. %.

Other useful tartar control agents include polymers and co-polymers. In some embodiments, the oral care compositions include one or more polymers, such as polyethylene glycols, polyvinyl methyl ether maleic acid copolymers, polysaccharides (e.g., cellulose derivatives, for example carboxymethyl cellulose, or polysaccharide gums, for example xanthan gum or carrageenan gum). Acidic polymers, for example polyacrylate gels, may be provided in the form of their free acids or partially or fully neutralized water-soluble alkali metal (e.g., potassium and sodium) or ammonium salts. Certain embodiments include 1:4 to 4:1 copolymers of maleic anhydride or acid with another polymerizable ethylenically unsaturated monomer, for example, methyl vinyl ether (methoxyethylene), having a molecular weight (M.W.) of about 30,000 to about 1,000,000, polyvinyl methyl ether/maleic anhydride (PVM/MA) copolymers such as GANTREZ® (e.g., GANTREZ® S-97 polymer). In some embodiments, the PVM/MA copolymer comprises a copolymer of methyl vinyl ether/maleic anhydride, wherein the anhydride is hydrolyzed following copolymerization to provide the corresponding acid. In some embodiments, PVM/MA copolymer has an average molecular weight (M.W.) of about 30,000 to about 1,000,000, e.g. about 300,000 to about 800,000, e.g., wherein the anionic polymer is about 1-5%, e.g., about 2%, of the weight of the composition. In some embodiments, the anti-calculus agent is present in the composition in an amount of from 0.2 weight % to 0.8 weight %; 0.3 weight % to 0.7 weight %; 0.4 weight % to 0.6 weight %; or about 0.5 weight %, based on the total weight of the composition. Copolymers are available for example as Gantrez AN 139(M.W. 500,000), AN 1 19 (M.W. 250,000) and S-97 Pharmaceutical Grade (M.W. 70,000), of GAF Chemicals Corporation. Other operative polymers include those such as the 1:1 copolymers of maleic anhydride with ethyl acrylate, hydroxyethyl methacrylate, N-vinyl-2-pyrollidone, or ethylene, the latter being available for example as Monsanto EMA No. 1 103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone. Suitable generally, are polymerized olefinically or ethyl enically unsaturated carboxylic acids containing an activated carbon-to-carbon olefinic double bond and at least one carboxyl group, that is, an acid containing an olefinic double bond which readily functions in polymerization because of its presence in the monomer molecule either in the alpha-beta position with respect to a carboxyl group or as part of a terminal methylene grouping. Illustrative of such acids are acrylic, methacrylic, ethacrylic, alpha-chloroacrylic, crotonic, beta-acryloxy propionic, sorbic, alpha-chlorsorbic, cinnamic, beta-styrylacrylic, muconic, itaconic, citraconic, mesaconic, glutaconic, aconitic, alpha-phenylacrylic, 2-benzyl acrylic, 2-cyclohexylacrylic, angelic, umbellic, fumaric, maleic acids and anhydrides. Other different olefinic monomers copolymerizable with such carboxylic monomers include vinylacetate, vinyl chloride, dimethyl maleate and the like. Copolymers contain sufficient carboxylic salt groups for water-solubility. A further class of polymeric agents includes a composition containing homopolymers of substituted acrylamides and/or homopolymers of unsaturated sulfonic acids and salts thereof, in particular where polymers are based on unsaturated sulfonic acids selected from acrylamidoalykane sulfonic acids such as 2-acrylamide 2 methylpropane sulfonic acid having a molecular weight of about 1,000 to about 2,000,000. Another useful class of polymeric agents includes polyamino acids, particularly those containing proportions of anionic surface-active amino acids such as aspartic acid, glutamic acid and phosphoserine.

In some embodiments, the oral care compositions comprise a saliva stimulating agent useful, for example, in amelioration of dry mouth. Any orally acceptable saliva stimulating agent can be used, including without limitation food acids such as citric, lactic, malic, succinic, ascorbic, adipic, fumaric and tartaric acids, and mixtures thereof. One or more saliva stimulating agents are optionally present in saliva stimulating effective total amount.

In some embodiments, the oral care compositions comprise a nutrient. Suitable nutrients include vitamins, minerals, amino acids, and mixtures thereof. Vitamins include Vitamins C and D, miamine, riboflavin, calcium pantothenate, niacin, folic acid, nicotinamide, pyridoxine, cyanocobalamin, para-aminobenzoic acid, bioflavonoids, and mixtures thereof. Nutritional supplements include amino acids (such as L-tryptophane, L-lysine, methionine, threonine, levocarnitine and L-carnitine), lipotropics (such as choline, inositol, betaine, and linoleic acid), and mixtures thereof.

In some embodiments, the oral care compositions comprise at least one viscosity modifier, useful for example to help inhibit settling or separation of ingredients or to promote re-dispersibility upon agitation of a liquid composition. Any orally acceptable viscosity modifier can be used, including without limitation, mineral oil, petrolatum, clays and organo-modified clays, silicas and the like. One or more viscosity modifiers are optionally present in a total amount of from about 0.01 wt. % to about 10 wt. %, for example, from about 0.1 wt. % to about 5 wt. %, by total weight of the composition.

In some embodiments, the oral care compositions comprise antisensitivity agents, e.g., potassium salts such as potassium nitrate, potassium bicarbonate, potassium chloride, potassium citrate, and potassium oxalate; capsaicin; eugenol; strontium salts; chloride salts and combinations thereof. Such agents may be added in effective amounts, e.g., from about 1 wt. % to about 20 wt. % by weight based on the total weight of the composition, depending on the agent chosen.

In some embodiments, the oral care compositions comprise an antioxidant. Any orally acceptable antioxidant can be used, including butylated hydroxy anisole (BHA), butylated hydroxytoluene (BHT), vitamin A, carotenoids, co-enzyme Q10, PQQ, Vitamin A, Vitamin C, vitamin E, anethole-dithiothione, flavonoids, polyphenols, ascorbic acid, herbal antioxidants, chlorophyll, melatonin, and mixtures thereof.

In some embodiments, the oral care compositions comprise of one or more alkali phosphate salts, e.g., sodium, potassium or calcium salts, e.g., selected from alkali dibasic phosphate and alkali pyrophosphate salts, e.g., alkali phosphate salts selected from sodium phosphate dibasic, potassium phosphate dibasic, dicalcium phosphate dihydrate, calcium pyrophosphate, tetrasodium pyrophosphate, tetrapotassium pyrophosphate, sodium tripolyphosphate, disodium hydrogenorthophoshpate, monosodium phosphate, pentapotassium triphosphate and mixtures of any of two or more of these, e.g., in an amount of 0.01-20%, e.g., 0.1-8%, e.g., e.g., 0.1 to 5%, e.g., 0.3 to 2%, e.g., 0.3 to 1%, e.g about 0.01%, about 0.1%, about 0.5%, about 1%, about 2%, about 5%, about 6%, by weight of the composition. In some embodiments, compositions comprise tetrapotassium pyrophosphate, disodium hydrogenorthophoshpate, monosodium phosphate, and pentapotassium triphosphate. In some embodiments, compositions comprise tetrasodium pyrophosphate from 0.1-1.0 wt % (e.g., about 0.5 wt %).

In some embodiments, the oral care compositions comprise a source of calcium and phosphate selected from (i) calcium-glass complexes, e.g., calcium sodium phosphosilicates, and (ii) calcium-protein complexes, e.g., casein phosphopeptide-amorphous calcium phosphate. Any of the preceding compositions further comprising a soluble calcium salt, e.g., selected from calcium sulfate, calcium chloride, calcium nitrate, calcium acetate, calcium lactate, and combinations thereof.

In some embodiments, the oral care compositions comprise an additional ingredient selected from: benzyl alcohol, Methylisothizolinone (“MIT”), Sodium bicarbonate, sodium methyl cocoyl taurate (tauranol), lauryl alcohol, and polyphosphate. Some embodiments comprise benzyl alcohol that is present from 0.1-0.8 wt %., or 0.2 to 0.7 wt %, or from 0.3 to 0.6 wt %, or from 0.4 to 0.5 wt %, e.g. about 0.1 wt. %, about 0.2 wt. %, about 0.3 wt %, about 0.4 wt %, about 0.5 wt %, about 0.6 wt %, about 0.7 wt % or about 0.8 wt %.

In some embodiments, the oral care compositions comprise from 5%-40%, e.g., 10%-35%, e.g., about 15%, 25%, 30%, and 35% or more of water.

Methods are provided for neutralizing toxicity of a lipopolysaccharide in an individual's oral cavity. The methods for neutralizing toxicity of a lipopolysaccharide in an individual's oral cavity may be performed on an individual suspected of or identified as having pathogenic gram-negative bacteria in their oral cavity which produces toxic lipopolysaccharide. In some embodiments, the individual is suspected of or identified as having one or more of the pathogenic gram-negative bacteria, Porphyromonas gingivalis, Escherichia coli, Prevotella intermedia, Fusobacterium nucleatum, Treponema denticola, Aggregatibacter actinomycetemcomitans or Tannerella forsythia. Other pathogenic bacteria, may include for example, Eikenella corrodens, Campylobacter rectus, Campylobacter gracilis, Streptococcus mutans, Streptococcus sobrinus, Streptococcus sanguis, Streptococcus oralis, Actinomyces israelii, Chlamydia pneumoniae, Porphyromonas cangingivalis, Fusobacterium necrophorum, and Streptococcus constellatus in their oral cavity. In some embodiments, the individual is identified as having one or more of the pathogenic gram-negative bacteria by obtaining a sample of bacteria from the individual's oral cavity and identifying the species present in the sample. In some embodiments, the individual is identified as having plaque and inflammation in the oral cavity. In some embodiments, the individual is identified as having plaque and inflammation within their gingival crevice. In some embodiments, the individual is identified as having plaque which comprises gram negative bacteria and inflammation in their oral cavity, such within their gingival crevice. In some embodiments, the method comprises the step of obtaining a sample of plaque from the individual and detecting one or more of the species of gram-negative bacteria, such as Porphyromonas gingivalis, Escherichia coli, Prevotella intermedia, Fusobacterium nucleatum, Treponema denticola, Aggregatibacter actinomycetemcomitans and Tannerella forsythia. In some embodiments, the individual is identified as having toxic lipopolysaccharide present in their oral cavity by detecting elevated levels of one or more proinflammatory cytokines, such as TNF-α, IL-6, IL-8, IL-1β and GM-CSF, in the individual's oral cavity. In some embodiments, the method comprises the step of obtaining a sample from the individual's oral cavity, such as a gingival fluid sample, and detecting elevated levels of one or more proinflammatory cytokines, such as TNF-α, IL-6, IL-8, IL-1β and GM-CSF, in the sample. In some embodiments, the individual is identified as having toxic lipopolysaccharide present in their oral cavity by detecting elevated levels of PGE2 in the individual's oral cavity. In some embodiments, the method comprises the step of obtaining a sample from the individual's oral activity, such as a sample of oral epithelial tissue, gingival epithelial tissue or gingival fluid, and detecting elevated levels of PGE2 in the sample. In some embodiments, the methods comprise the step of establishing a baseline level of one or more proinflammatory cytokines, such as TNF-α, IL-6, IL-8, IL-1β and GM-CSF, or of establishing a baseline level of PGE2 by obtaining from the oral cavity of an individual that does not have toxic lipopolysaccharide present in their oral cavity, a sample, and detecting the level of one or more proinflammatory cytokines, such as TNF-α, IL-6, IL-8, IL-1β and GM-CSF, or of PGE2, present in the sample. The baseline level is recorded and used as a reference in future assays in which a sample from the individual's oral cavity and levels of one or more proinflammatory cytokines, such as TNF-α, IL-6, IL-8, IL-1β and GM-CSF, or of PGE2, in the sample are detected. If the levels detected are greater than the baseline levels, the individual has elevated levels of the one or more proinflammatory cytokines or PGE2 and the presence of toxic lipopolysaccharide is indicated. In some embodiments, the methods of neutralizing toxicity of a lipopolysaccharide in an individual's oral cavity may comprise the steps of identifying the individual toxicity of a lipopolysaccharide in an individual's oral cavity by the steps described above and administering to the oral cavity of the individual an oral care composition, such as a toothpaste, comprising zinc oxide and zinc citrate, and optionally, fluoride and/or arginine, in an amount effective to inhibit secretion of one or more proinflammatory cytokines and/or prostaglandin E2 by cells of the individual. In some embodiments, the zinc oxide may be present in an amount of from 0.75 to 1.25 wt % based on the total weight of the composition, and the zinc citrate is present in an amount of from 0.25 to 1.0 wt % based on the total weight of the composition. In some embodiments, the ratio of the amount of zinc oxide by wt % to zinc citrate by wt % is 2:1, 2.5:1, 3:1, 3.5:1 or 4:1, based on the total weight of the composition. In some embodiments, arginine is present in an amount of from 0.1% to 15%, and in some embodiments from 0.5% to 3%, based on the total weight of the composition, the weight of the basic amino acid being calculated as free form. Arginine, which in some embodiments is L-arginine, may be in free form or salt form. In some embodiments, stannous fluoride is present such as in an amount of 0.1 wt, % to 2 wt. % based on the total weight of the composition.

### EXAMPLES

### Example 1

Commercially available HEK-Blue TLR4 cells (Invitrogen) are recombinant HEK 293 cells that have been transformed with DNA that encodes TLR4 that is expressed and present on the cell surface. HEK-Blue TLR4 cells are grown ion culture and used can be used in experimental assays to identify test compositions that modulate inflammation pathways stimulated by TLR4-LPS interaction.

In addition to the HEK-Blue hTLR4 cells, equipment and supplied used in the growing, culturing and use of the cells in experimental assays include: DMEM, HEK-Blue Selection 250× reagent, Normocin, FBS, Penicillin-streptomycin, a sterile tissue culture hood and an incubator at 370 Celsius, 5% CO₂.

Media for starting cells (starting media) from storage should not contain HEK-Blue Selection. Media is prepared by aspirating 56 mL of DMEM from a 500 mL bottle. To the remaining DMEM, 50 mL of FBS, 5 mL of Penicillin-streptomycin, and 1 mL of Normocin are added to make DMEM supplemented with 10% FBS, 1% Penicillin-streptomycin, and 100 ug/mL Normocin.

Media for culturing cells (culturing media) after they have been established from frozen stock contains HEK-Blue Selection agent. Media is prepared by aspirating 58 mL of DMEM from a 500 mL bottle. To the remaining DMEM, 50 mL of FBS, 5 mL of Penicillin-streptomycin, 1 mL of Normocin, and 2 mL of 250×HEK-Blue Selection agent are added to make DMEM supplemented with 10% FBS, 1% Penicillin-streptomycin, 100 ug/mL Normocin, with 1×HEK-Blue Selection.

To initially culture the cells, the cells are first thawed immediately upon receipt. To thaw the cells, the vial contained the cells is placed in a 37° C. water bath. To prevent contamination, the cap of the vial is kept out of the water. Upon thawing, the vial is removed from the water bath, sprayed with 70% ethanol and moved into tissue culture hood. The cells are resuspended in a vial of 5-15 mL of starting media and centrifuge at 1000 RPM for five minutes. The supernatant is aspirated, taking care not to aspirate the pellet of cells at the bottom of the vial. The pellet of cells is resuspended in starting media, then transferred into a tissue culture flask. The cells in the tissue culture claims are incubate at 37° C. and 5% CO₂. Once cells are established, they can be passaged as detailed below using culturing media.

To culture and passaging the cells, media is renewed every other day, or at least twice a week, using culturing media (i.e. media with HEK-Blue selection agent). Cells are split when approximately 70-80% confluent. A cell scraper may be used to detach the cells from the flask. Alternatively, media in the flask may be aspirated and PBS is added to the flask. The flask is tapped to detach cells. To reduce clumping, the detached cells are pipetted gently up and down. Once collected, cells are centrifuge at 1000 RPM for 5 minutes. The supernatant is aspirated and the pellet containing the cells may be resuspended in media and transferred into a tissue culture flask, which is incubated at 37° C. and 5% CO₂.

Cells may be frozen for storage and future use. To do so, a 10% DMSO solution diluted with DMEM media is prepared. Following the steps above for passaging cells, cells are detached from the flask, and collected and centrifuged. After aspirating supernatant, the cells in the pellet are resuspended using the 10% DMSO solution. 1 mL of the cell suspension is aliquoted into cryogenic vials which are frozen at −80° C. overnight and then transfer to liquid nitrogen.

Testing solutions were made as follows:

Initial for LPS and zinc-containing starting solutions were made as follows. An LPS starting solution was prepared as follows: a 0.2 ag/ml solution of LPS was prepared using 2 μl of 1 mg/ml LPS and 10 ml media. Multiple zinc starting solutions were each prepared using media as diluent: a) 20 mM zinc oxide, b) 2 mM zinc oxide, c) 20 mM zinc citrate, d) 2 mM zinc citrate, e) 20 mM of a composition containing a 2:1 mixture of zinc oxide and zinc citrate and f) 2 mM of a composition containing a 2:1 mixture of zinc oxide and zinc citrate. The starting solutions were used to prepare various test samples.

In some embodiments, control assays include “Untreated”, in which eight Zinc test samples without LPS were made: 10 mM zinc oxide, 1 mM zinc oxide, 10 mM zinc citrate, 1 mM zinc citrate, 10 mM zinc oxide+zinc citrate, 1 mM zinc oxide+zinc citrate, 10 mM zinc oxide+zinc citrate+arginine and 1 mM zinc oxide+zinc citrate+arginine, The test samples without LPS were prepared by diluting the zinc starting solutions with equal parts media. Eight test samples with LPS were made: 10 mM zinc oxide LPS, 1 mM zinc oxide LPS, 10 mM zinc citrate LPS, 1 mM zinc citrate LPS, 10 mM zinc oxide+zinc citrate LPS, 1 mM zinc oxide+zinc citrate LPS, 10 mM zinc oxide+zinc citrate+arginine LPS and 1 mM zinc oxide+zinc citrate+arginine LPS. The test samples with LPS were prepared by diluting the zinc starting solutions with equal parts LPS starting solution.

Experiments were performed as follows.

Cells were grown to 70-80% confluent in a 96 well plate. 100 uL of a test sample was added to a well. Cells with test sample were incubated overnight. Media was collected from each well and transferred to a corresponding well in a 96-well plate. Assays to measure proinflammatory cytokines including IL-8 secretion were performed for each well.

Inflammation markers in supernatants collected from HEK-Blue cells co-incubated with Zinc and Zinc+LPS were quantified using Luminex Magpix instrument (MAGPIX-XPON42) and human 5-plex cytokine/chemokine Magnetic bead panel (Millipore HCYTOMAG-60K). The human 5-plex cytokine/chemokine Magnetic bead panel quantifies TNF-α, IL-6, IL-8, IL-1β and GM-CSF.

The Luminex Kit Procedure outlined in the instruction manual for each separate Luminex kit was performed on the supernatants collected from the HEK-Blue cells co-incubated with Zinc test solutions without LPS and Zinc test solutions plus LPS. Quality controls in range for the specified analytes were performed to ensure levels were within the acceptable range.

To run the Luminex Test, all necessary calibration and verification procedures as outlined by the MagPix and xPonent softwares were completed and the appropriate protocol for the assay was selected. The standards were verified to be correct and complete the plate layout of samples was verified. The program was run to completion and results were displayed in a .csv file that could be opened using Microsoft Excel. The “Avg. Result” was identified in the results generated.

### Example 2

Experiments were conducted to identify compositions that inhibit P. gingivalis LPS-induced IL-8, TNFα, GM-CSF, IL-1β and IL-6 by HEK-TLR4 cells. HEK-TLR4 cells in 96 well plates were contacted with 100 μl samples. IL-8, TNFα, GM-CSF, IL-1β and IL-6 were detected as described above. Data in Table 1 shows average results from experiments measuring IL-8 (pg/ml) using Untreated cells (UNT), cells treated with 0.1μ/ml P. gingivalis LPS (LPS), cells treated with 300 ppm zinc oxide (ZnO 300), cells treated with 600 ppm zinc oxide (ZnO 600), cells treated with 300 ppm zinc citrate (ZnC 300), cells treated with 600 ppm zinc citrate (ZnC 600), cells treated with 300 ppm of a mixture of zinc oxide and zinc citrate in a ratio of about 2:1 by % weight (MIXA 300; collectively 300 ppm-approx. 281 ppm ZnO, 19 ppm ZnC); cells treated with 600 ppm of a mixture of zinc oxide and zinc citrate in a ratio of about 2:1 by % weight (MIXA 600; collectively 600 ppm-approx. 562 ppm ZnO, 38 ppm ZnCitrate); cells treated with a solution containing 300 ppm of a mixture of zinc oxide, zinc citrate and arginine in a ratio of about 2:1:3 by % weight (MIXB 300; collectively 300 ppm-approx. 167 ppm ZnO, 11 ppm ZnC, 122 ppm arginine); cells treated with a solution containing 600 ppm of a mixture of zinc oxide, zinc citrate and arginine in a ratio of about 2:1:3 by % weight (MIXB 600; collectively 600 ppm-approx. 334 ppm ZnO, 22 ppm ZnC, 244 ppm arginine); cells treated with a solution containing 0.1μ/ml P. gingivalis LPS and 300 ppm zinc oxide (ZnO LPS 300), cells treated with a solution containing 0.1μ/ml P. gingivalis LPS and 600 ppm zinc oxide (ZnO LPS 600), cells treated with a solution containing 0.1μ/ml P. gingivalis LPS and 300 ppm zinc citrate (ZnC LPS 300), cells treated with a solution containing 0.1μ/ml P. gingivalis LPS and 600 ppm zinc citrate (ZnC LPS 600), cells treated with a solution containing 0.1μ/ml P. gingivalis LPS and 300 ppm MIXA (MIXA LPS 300; 0.1μ/ml P. gingivalis LPS, approx. 281 ppm ZnO, 19 ppm ZnC), cells treated with a solution containing 0.1μ/ml P. gingivalis LPS and 600 ppm MIXA (MIXA LPS 600; 0.1μ/ml P. gingivalis LPS, approx. 562 ppm ZnO, 38 ppm ZnC), cells treated with a solution containing 0.1μ/ml P. gingivalis LPS and 300 ppm MIXB (MIXB LPS 300; 0.1μ/ml P. gingivalis LPS, approx. 167 ppm ZnO, 11 ppm ZnC, 122 ppm arginine), and cells treated with a solution containing 0.1μ/ml P. gingivalis LPS and 600 ppm MIXB (MIXB LPS 600; 0.1μ/ml P. gingivalis LPS, approx. 334 ppm ZnO, 22 ppm ZnCitrate, 244 ppm arginine).

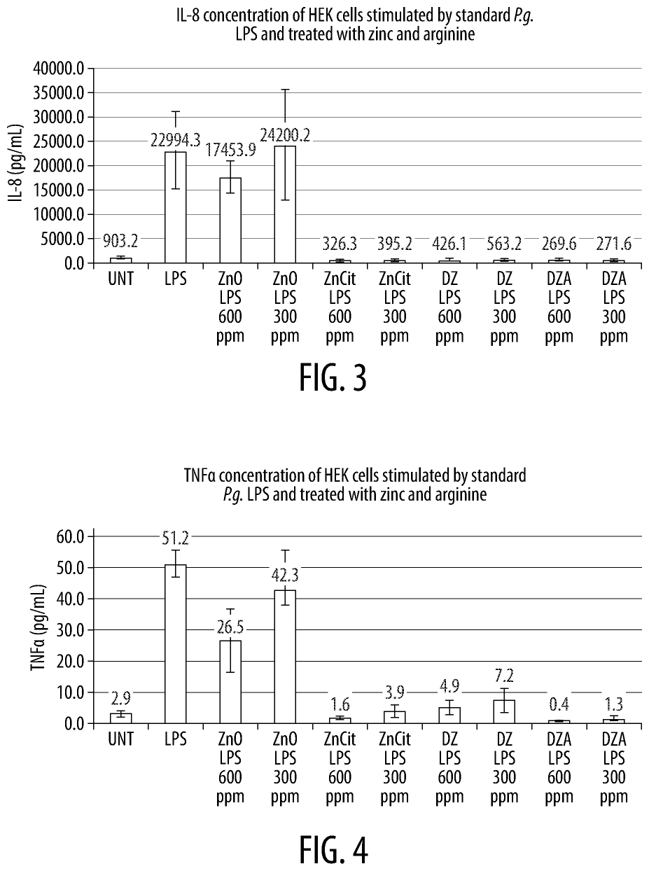

FIG. 3 and Table 1 show data generated measuring

TABLE 1

| Sample | IL-8 pg/ml |
|---|---|
| Untreated | 903.22608235438 |
| LPS | 22994.30589404500 |
| ZnO 600 | 558.782498450305 |
| ZnO 300 | 452.783115432497 |
| ZnC 600 | 293.938960166149 |
| ZnC 300 | 293.972340074672 |
| MIXA 600 | 314.515734623946 |
| MIXA 300 | 290.785725526731 |
| MIXB 600 | 277.870635034924 |
| MIXB 300 | 284.34412329375 |
| ZnO LPS 600 | 17453.90185032290 |
| ZnO LPS 300 | 24200.24439257880 |
| ZnC LPS 600 | 326.319953087408 |
| ZnC LPS 300 | 395.214416510412 |
| MIXA LPS 600 | 426.135145414651 |
| MIXA LPS 300 | 563.160327194025 |
| MIXB LPS 600 | 269.550657539641 |
| MIXB LPS 300 | 271.605870558933 |

The data demonstrate that the presence of zinc oxide had essentially a comparatively small effect on induction of IL-8 by LPS compared to zinc citrate which showed profound inhibition LPS-induced IL-8. The combination of zinc oxide and zinc citrate in MIXA showed the inhibitory effect more than additive. The combination of zinc oxide, zinc citrate and arginine in MIXB showed an increased inhibitory effect relative to the inhibitory effect of the combination with zinc oxide and zinc citrate in MIXA.

FIG. 4 and Table 2 show data generated measuring TNF-α.

TABLE 2

| Sample | TNF-α pg/mL |
|---|---|
| Untreated | 2.883914621209270 |
| LPS | 51.197345751888300 |
| ZnO 600 | 2.404656713944550 |
| ZnO 300 | 1.202310842820140 |
| ZnC 600 | 1.172475285567080 |
| ZnC 300 | 1.616244942132960 |
| MIXA 600 | 1.337356182121090 |
| MIXA 300 | 1.589119338050950 |
| MIXB 600 | 0.466059799174690 |
| MIXB 300 | 0.904697489751425 |
| ZnO LPS 600 | 26.473342911222500 |
| ZnO LPS 300 | 42.302328747816800 |
| ZnC LPS 600 | 1.596207555984190 |
| ZnC LPS 300 | 3.857078297259910 |
| MIXA LPS 600 | 4.881910070574060 |
| MIXA LPS 300 | 7.221046104652260 |
| MIXB LPS 600 | 0.415826464983407 |
| MIXB LPS 300 | 1.32433286941780 |

The data demonstrate that the presence of zinc oxide reduced LPS-induced TNFα. Zinc citrate showed more profound inhibition LPS-induced TNFα. The combination of zinc oxide and zinc citrate in MIXA showed the inhibitory effect more than additive. The combination of zinc oxide, zinc citrate and arginine in MIXB showed an increased inhibitory effect relative to the inhibitory effect of the combination with zinc oxide and zinc citrate in MIXA.

The Average Result data shown in Table 1 and Table 2 are based upon the raw data shown in Table 3 which includes the standard deviation of the data generated for the IL-8 data.

TABLE 3

| Sample | IL-8 pg/ml | Standard deviation | TNFα pg/ml |
|---|---|---|---|
| Untreated | 1008.865094114080 | 234.695340529898 | 4.12632419694976 |
| Untreated | 577.912257826429 | | 1.58827962206202 |
| Untreated | 1122.900895122630 | | 2.93714004461605 |
| LPS | 15024.043431805100 | 8140.08851457577 | 54.4178287953412 |
| LPS | 34173.027935737200 | | 54.2247204189207 |
| LPS | 19785.846314592700 | | 44.9494880414031 |
| ZnO 600 | 993.279402711237 | 258.096326375729 | 3.72874524237378 |
| ZnO 600 | 512.486811241409 | | 2.19474090368396 |
| ZnO 600 | 355.356874434547 | | 1.80427367395076 |
| ZnO 600 | 374.006905414025 | | 1.89086703576971 |
| ZnO 300 | 340.203048180867 | 104.790829947577 | 0.902890933610745 |
| ZnO 300 | 393.091973128309 | | 1.03059565432327 |
| ZnO 300 | 458.767352999561 | | 1.24433691360079 |
| ZnO 300 | 619.070087421251 | | 1.63141986974574 |
| ZnC 600 | 434.484832646520 | 81.7364081108077 | 2.0643634588267 |
| ZnC 600 | 262.068108192788 | | 1.07325840719104 |
| ZnC 600 | 234.634653885537 | | 0.606869293824337 |
| ZnC 600 | 244.568245939749 | | 0.945409982426227 |
| ZnC 300 | 254.640608322407 | 57.8442169161337 | 1.24433691360079 |
| ZnC 300 | 237.462254415899 | | 1.67459009841178 |
| ZnC 300 | 296.889849817204 | | 1.37305598924565 |
| ZnC 300 | 386.896647743178 | | 2.1729967672736 |
| MIXA 600 | 469.837778126971 | 94.0501422752389 | 2.08607828741614 |
| MIXA 600 | 309.031689652221 | | 1.00928141288852 |
| MIXA 600 | 240.301078609214 | | 0.945409982426227 |
| MIXA 600 | 238.892392107377 | | 1.30865504575347 |
| MIXA 300 | 231.986416050415 | 64.4358339197553 | 1.3301130115629 |
| MIXA 300 | 254.491833569850 | | 1.39454073708412 |
| MIXA 300 | 277.867696483195 | | 1.48056522002557 |
| MIXA 300 | 398.796956003465 | | 2.15125838353123 |
| MIXB 600 | 466.669789022714 | 112.0210169421000 | 1.28720627824567 |
| MIXB 600 | 252.164987275957 | | 0.356111746347439 |
| MIXB 600 | 179.160167480587 | | 0.0702395430162487 |
| MIXB 600 | 213.487596360440 | | 0.150681629089399 |
| MIXB 300 | 229.779078358643 | 81.4961070558465 | 0.860423946154894 |
| MIXB 300 | 240.519916087771 | | 0.733360659614878 |
| MIXB 300 | 241.809980358633 | | 0.522913226032664 |
| MIXB 300 | 425.267518369955 | | 1.50209212720327 |
| ZnO LPS 600 | 21030.114488079100 | 3273.1767741231900 | 43.9931887732765 |
| ZnO LPS 600 | 14514.694724312700 | | 24.753819906264 |
| ZnO LPS 600 | 20392.976523123200 | | 19.1279980781763 |
| ZnO LPS 600 | 13877.821665776500 | | 18.018364887173 |
| ZnO LPS 300 | 31890.081340469700 | 11546.0267130803000 | 41.5589183468906 |
| ZnO LPS 300 | 39006.703793117100 | | 39.7968079484333 |
| ZnO LPS 300 | 13910.330133842900 | | 38.156800336051 |
| ZnO LPS 300 | 11993.862302885500 | | 49.6967883598923 |
| ZnC LPS 600 | 447.329900328848 | 70.0220221839775 | 2.58706377690339 |
| ZnC LPS 600 | 287.811391318559 | | 1.41603415478221 |
| ZnC LPS 600 | 291.502704203514 | | 1.26576685174274 |
| ZnC LPS 600 | 278.635816498712 | | 1.11596544050842 |
| ZnC LPS 300 | 364.456715467181 | 93.8694383786809 | 3.11260586971185 |
| ZnC LPS 300 | 386.837866749303 | | 2.69633455863665 |
| ZnC LPS 300 | 285.429428876610 | | 2.32532366370544 |
| ZnC LPS 300 | 544.133654948556 | | 7.2940490969857 |
| MIXA LPS 600 | 716.160967218628 | 167.7855498556630 | 8.82627701197471 |
| MIXA LPS 600 | 318.420486878204 | | 3.68463838798152 |
| MIXA LPS 600 | 323.320707516375 | | 3.55240392497041 |
| MIXA LPS 600 | 346.638420045396 | | 3.46432095736961 |
| MIXA LPS 300 | 384.343530518389 | 226.6378659716090 | 4.54712231202829 |
| MIXA LPS 300 | 470.291277313394 | | 5.14691560464427 |
| MIXA LPS 300 | 446.068296027251 | | 4.99121970160287 |
| MIXA LPS 300 | 951.938204917066 | | 14.1989268003336 |
| MIXB LPS 600 | 408.091070201322 | 82.0401700547990 | 0.945409982426227 |
| MIXB LPS 600 | 249.993389968937 | | 0.314696031975567 |
| MIXB LPS 600 | 221.633430489907 | | 0.19138121710145 |
| MIXB LPS 600 | 198.484739498398 | | 0.211818628430383 |
| MIXB LPS 300 | 226.242512246204 | 63.6871803695681 | 0.860423946154894 |
| MIXB LPS 300 | 261.291931511083 | | 1.15871516806729 |
| MIXB LPS 300 | 220.355849263928 | | 0.691128586545636 |
| MIXB LPS 300 | 378.533189214519 | | 2.58706377690339 |

Data in Table 4 is raw data from measuring GM-CSF, IL-1β and IL6 (pg/ml for each, respectively).

TABLE 4

| Sample | GM-CSF | IL-1β | IL-6 |
|---|---|---|---|
| Untreated | 0.0725299329866643 | 2.4238203474115 | 1.47783331744434 |
| Untreated | 0.0725299329866643 | 1.38990929536491 | 1.04287084524682 |
| Untreated | 0.386312226008842 | 3.04969278746046 | 1.66113094758605 |
| LPS | 1.05596871181312 | 5.51938548485799 | 4.73908750721215 |
| LPS | 1.05596871181312 | 6.36974005089928 | 3.77539445536265 |
| LPS | 1.57685185247333 | 6.18727175284026 | 3.37338771069631 |
| ZnO 600 | 0.885354184438006 | 2.8705269344633 | 1.01132966093281 |
| ZnO 600 | 0.226306914299891 | 1.03912352976383 | 0.324823691344939 |
| ZnO 600 | 0.00167131422604622 | 0.432860388135171 | <3.2 |
| ZnO 600 | 0.00167131422604622 | 0.404352172353604 | <3.2 |
| ZnO 300 | <3.2 | 0.461412975927705 | 0.460533924013502 |
| ZnO 300 | <3.2 | 0.461412975927705 | 0.658729151093581 |
| ZnO 300 | 0.305745618569087 | 0.864729870839965 | 0.979714063030776 |
| ZnO 300 | 0.550075822413103 | 1.50733640051162 | 1.29280029481962 |
| ZnC 600 | 0.148336958881236 | 0.922776218419179 | 0.324823691344939 |
| ZnC 600 | 0.0725299329866643 | 0.290829704914395 | <3.2 |
| ZnC 600 | <3.2 | 0.0138130330649466 | <3.2 |
| ZnC 600 | <3.2 | 0.122716996836715 | <3.2 |
| ZnC 300 | <3.2 | 0.0138130330649466 | <3.2 |
| ZnC 300 | <3.2 | 0.122716996836715 | <3.2 |
| ZnC 300 | <3.2 | 0.234438673343891 | <3.2 |
| ZnC 300 | <3.2 | 0.404352172353604 | <3.2 |
| MIXA 600 | 0.00167131422604622 | 0.980909061173732 | 0.255412222436987 |
| MIXA 600 | <3.2 | 0.234438673343891 | <3.2 |
| MIXA 600 | <3.2 | 0.122716996836715 | <3.2 |
| MIXA 600 | <3.2 | 0.0676743990810237 | <3.2 |
| MIXA 300 | <3.2 | <3.2 | <3.2 |
| MIXA 300 | <3.2 | 0.122716996836715 | <3.2 |
| MIXA 300 | <3.2 | 0.234438673343891 | <3.2 |
| MIXA 300 | 0.148336958881236 | 0.691168798941226 | 0.184524608110801 |
| MIXB 600 | 0.305745618569087 | 1.03912352976383 | <3.2 |
| MIXB 600 | <3.2 | 0.0138130330649466 | <3.2 |
| MIXB 600 | <3.2 | <3.2 | <3.2 |
| MIXB 600 | <3.2 | <3.2 | <3.2 |
| MIXB 300 | <3.2 | <3.2 | <3.2 |
| MIXB 300 | <3.2 | <3.2 | <3.2 |
| MIXB 300 | <3.2 | <3.2 | <3.2 |
| MIXB 300 | <3.2 | 0.748919428749724 | <3.2 |
| ZnO LPS 600 | 1.14189633052164 | 6.73506169804816 | 2.61670689303483 |
| ZnO LPS 600 | 1.31487100974257 | 6.30890274203823 | 2.43987975132065 |
| ZnO LPS 600 | 0.386312226008842 | 6.03531613368765 | 1.7824761203747 |
| ZnO LPS 600 | 0.305745618569087 | 5.70134871198118 | 2.02335215553719 |
| ZnO LPS 300 | 0.550075822413103 | 6.49145768238289 | 3.31567292299977 |
| ZnO LPS 300 | 0.633035686618357 | 6.49145768238289 | 3.77539445536265 |
| ZnO LPS 300 | 0.550075822413103 | 5.94418788212014 | 3.17105195858638 |
| ZnO LPS 300 | 1.57685185247333 | 6.67413988690441 | 4.17420832326732 |
| ZnC LPS 600 | <3.2 | 0.633531993084581 | <3.2 |
| ZnC LPS 600 | <3.2 | 0.122716996836715 | <3.2 |
| ZnC LPS 600 | <3.2 | 0.234438673343891 | <3.2 |
| ZnC LPS 600 | <3.2 | 0.122716996836715 | <3.2 |
| ZnC LPS 300 | <3.2 | 0.404352172353604 | <3.2 |
| ZnC LPS 300 | <3.2 | 0.518641090819971 | <3.2 |
| ZnC LPS 300 | <3.2 | 0.122716996836715 | <3.2 |
| ZnC LPS 300 | 0.633035686618357 | 1.33128266142379 | 0.593242645562038 |
| MIXA LPS 600 | 0.00167131422604622 | 1.38990929536491 | 0.39312350661471 |
| MIXA LPS 600 | <3.2 | 0.150481769983168 | <3.2 |
| MIXA LPS 600 | <3.2 | 0.178368409875459 | <3.2 |
| MIXA LPS 600 | <3.2 | 0.347481190378949 | <3.2 |
| MIXA LPS 300 | <3.2 | 0.347481190378949 | <3.2 |
| MIXA LPS 300 | 0.00167131422604622 | 0.691168798941226 | <3.2 |
| MIXA LPS 300 | 0.00167131422604622 | 0.691168798941226 | 0.0341352983996712 |
| MIXA LPS 300 | 0.716606058191713 | 2.27533815162795 | 0.852448775670767 |
| MIXB LPS 600 | <3.2 | 0.518641090819971 | <3.2 |
| MIXB LPS 600 | <3.2 | <3.2 | <3.2 |
| MIXB LPS 600 | <3.2 | <3.2 | <3.2 |
| MIXB LPS 600 | <3.2 | <3.2 | <3.2 |
| MIXB LPS 300 | <3.2 | <3.2 | <3.2 |
| MIXB LPS 300 | <3.2 | 0.122716996836715 | <3.2 |
| MIXB LPS 300 | <3.2 | <3.2 | <3.2 |
| MIXB LPS 300 | 0.386312226008842 | 0.748919428749724 | 0.184524608110801 |

Two pro-inflammatory cytokines, interleukin 8 (CXC-8 or IL-8) and tumor necrosis factor alpha (TNFα˜), were significantly reduced in the presence of zinc citrate, zinc oxide and combination of the three compounds at various doses. Through multiple experiments described here and in Examples below, the data show that the majority of the reduction in inflammation is contributed by the zinc compounds and the effect is retained in the presence of arginine.

### Example 3

Experiments were conducted to identify compositions that inhibit E. coli LPS-induced IL-8 and P. gingivalis LPS-induced IL-8 by HEK-Blue hTLR4 cells. HEK-TLR4 cells in 96 well plates were contacted with 100 μl samples. IL-8 was detected as described above. Data in Table 5 shows average results from experiments measuring IL-8 (pg/ml) using Untreated cells, cells treated with a solution containing 1.0 μg/ml E. coli LPS (E. coli LPS), cells treated with a solution containing 1.0 μg/ml P. gingivalis LPS (PG LPS), cells treated with a solution containing 500 ppm zinc oxide (ZnO 500), cells treated with a solution containing 100 ppm zinc oxide (ZnO 100), cells treated with a solution containing 500 ppm of a mixture of zinc oxide and zinc citrate (MIXC 500; total zinc compounds collectively 500 ppm), cells treated with a solution containing 100 ppm of a mixture zinc oxide and zinc citrate (MIXC; total zinc compounds collectively 100 ppm), cells treated with 1.0 μg/ml E. coli LPS and either 500 ppm or 100 ppm zinc oxide (ZnO E. coli 500 and ZnO E. coli 100), cells treated with 1.0 μg/ml P. gingivalis LPS and either 500 ppm or 100 ppm zinc oxide (ZnO PG 500 and ZnO PG 100), and cells treated with 1.0 μg/ml P. gingivalis LPS and either 500 ppm or 100 ppm of MIXC (MIXC E. coli 500 and MIXC E. coli 100), and cells treated with 1.0 μg/ml P. gingivalis LPS and either 500 ppm or 100 ppm of MIXC (MIXC PG 500 and MIXC PG 100).

TABLE 5

| Sample | IL-8 pg/ml |
|---|---|
| Untreated | 378.390134 |
| E. coli LPS | 1746308145 |
| P. gingivalis LPS | 2657020448 |
| ZnO 500 | 448.55393 |
| ZnO 100 | 610.13874 |
| MIXC 500 | 291.13915 |
| MIXC 100 | 2427.6622 |
| ZnO Ecoli 500 | 564963251 |
| ZnO Ecoli 100 | 317267829 |
| ZnO PG 500 | 275576697 |
| ZnO PG 100 | 210374063 |
| MIXC Ecoli 500 | 519.93127 |
| MIXC Ecoli 100 | 128319579 |
| MIXC PG 500 | 398.41119 |
| MIXC PG 100 | 40271728 |

The data demonstrate that the presence of zinc oxide induction of IL-8 by E. coli LPS at both the 500 ppm and 100 ppm levels. Zinc oxide also showed an inhibitory effect of P. gingivalis LPS at both the 500 ppm and 100 ppm levels. The combination of zinc oxide and zinc citrate showed a higher level of inhibitory effect of E. coli LPS and P. gingivalis LPS compared to ZnO.

### Example 4

Objective

There are many different ways to block TLR4 signaling, including blocking the LPS binding site, binding LPS to sequester it, or blocking the kinase cascade signaling pathway. Data was reported indicating that the treatment of human gingival fibroblasts with zinc chloride had decreased PGE₂ output than control when stimulated by Phorbol-12-myristate-13-acetate (PMA) but that IL-8 decreased in zinc chloride-treated cells compared to control. The possible role of zinc in blocking the LPS binding site as well as interfering with the kinase activity was further evaluated. The objective of this study is to assess the role of zinc oxide, zinc citrate and arginine and compositions that contain a combination of zinc oxide, zinc citrate and arginine in inhibiting inflammation via NFκB activation induced by bacterial endotoxins bound to human TLR4 receptors. The role of the individual zinc component and whether arginine plays a significant role in this pathway were also determined. Pro-inflammation cytokine panels were investigated in post-treatment cellular supernatants in the absence and presence of LPS and zinc or arginine or the mixture.

Materials and Methods

Monolayer Cell Treatment

Cell Culture

HEK-Blue hTLR4 cells (Invivogen, cat #hkb-htlr4) were cultured in a DMEM culture medium supplemented with 10% FBS and 1% penstrep at 37° C. with 5% CO₂. LPS from Porphyromonas gingivalis (P.g. LPS; Invivogen, cat #tlrl-pglps and cat #tlrl-ppglps) activates TLR4 receptor and downstream inflammation in HEK-Blue hTLR4 cells. P.g. LPS is co-incubated with HEK-Blue hTLR4 cells in the absence or presence of the simple solutions. Standard and ultrapure P.g. LPS were both used due to ultrapure being a specific ligand for the TLR4 receptor while standard LPS is a ligand for both TLR2 and TLR4. The HEK-Blue hTLR4 cells are specific for TLR4 and after troubleshooting it was determined that ultrapure P.g. LPS most effectively stimulates HEK-hTLR4 cells.

Test Samples

The simple solutions a) Zinc Oxide (ZnO), b) Zinc Citrate (ZnCitrate), c) Zinc Oxide+Zinc Citrate (Dual Zinc; DZ), d) Dual Zinc+Arginine (DZA, zinc oxide, zinc citrate and arginine), and e) L-Arginine (Arg) were diluted at different concentrations with culture medium.

Two sets of experiments are reported here:

1) experiments using 1 mM vs 10 mM total zinc; and

2) experiments using 3×, 6×, 12×, and 24× dilution fold of the simple solutions (see Table 6 below).

TABLE 6
Simple solution preparation of Zinc Oxide, Zinc Citrate, Arginine and Mixtures.

| | Dilution Factors | | | | |
|---|---|---|---|---|---|
| Sample | Stock | 3X | 6X | 12X | 24X |
| ZnO | 122 mM | 40.7 mM | 20.3 mM | 10.2 mM | 5.1 mM |
| ZnCitrate | 17 mM | 5.7 mM | 2.8 mM | 1.4 mM | 0.7 mM |
| DZ | 122 mM ZnO + | 40.7 mM ZnO + | 20.3 mM ZnO + | 10.2 mM ZnO + | 5.1 mM ZnO + |
| | 17 mM | 5.7 mM | 2.8 mM | 1.4 mM | 0.7 mM |
| | ZnCitmte | ZnCitrate | ZnCitrate | ZnCitmte | ZnCitrate |
| DZA | 122 mM ZnO + | 40.7 mM ZnO + | 20.3 mM ZnO + | 10.2 mM ZnO + | 5.1 mM ZnO + |
| | 17 mM | 5.7 mM | 2.8 mM | 1.4 mM | 0.7 mM |
| | ZnCitmte + | ZnCitrate + | ZnCitrate + | ZnCitmte + | ZnCitrate + |
| | 86 mM Arg | 28.7 mM Arg | 14.3 mM Arg | 7.2 mM Arg | 3.6 mM Arg |
| Arg | 86 mM | 28.7 mM | 14.3 mM | 7.2 mM | 3.6 mM |

Procedures:

HEK-Blue hTLR4 cells were plated in 96-well plates and grown until confluent at 37° C. and 5% CO₂ in HEK-blue selection media (DMEM with 10% FBS, 1% penicillin-streptomycin, and antibiotics for HEK-blue selection (Invivogen, cat #hb-sel)). Cells were co-incubated overnight with varying concentrations of different zinc actives as well as arginine and stimulated with either 0.1 μg/mL standard P. gingivalis LPS or 1 μg/mL ultrapure P. gingivalis LPS. Cell supernatants were collected for human inflammation cytokine panels using Multiplex and ELISA analysis.

Sample Analysis

Multiplex analysis was performed using the MILLIPLEX MAP Human Cytokine/Chemokine Magnetic Bead Panel—Immunology Multiplex Assay for IL-1, IL-6, IL-8, TNFα, and GM-CSF (Millipore Sigma, cat #HCYTOMAG-60K). Multiplex analysis was used to first identify IL-8 and TNFα as the leading pro-inflammatory cytokines. IL-8 analysis was performed using the Enzo IL-8 ELISA assay (Enzo Life Sciences, cat #ADI-901-156A) as it was the best candidate for the pro-inflammatory cytokine produced in response to P.g. LPS stimulation.

Results

Testing was performed using total zinc concentrations to normalize the amount the zinc applied in the cell culture medium during co-incubation. Two concentrations of total zinc ion are based on the potential amount of zinc retained on the mouth soft tissue surface.

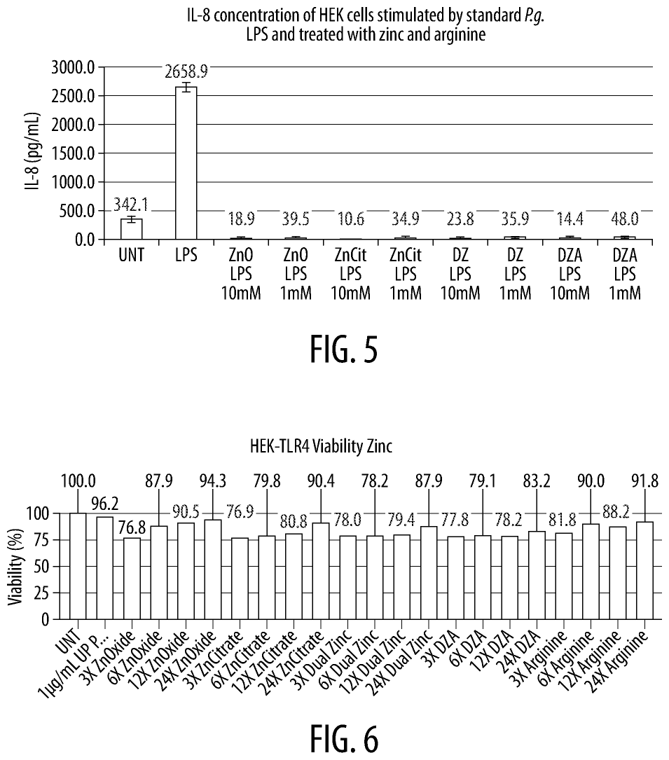

Date is shown in FIG. 5 and Table 7.

As shown in FIG. 5, the inflammation cytokine IL-8 induced by co-incubation with P.g. LPS was reduced by ZnO, ZnCitrate, DZ and DZA at both 1 mM and 10 mM.

TABLE 7
Concentration of IL-8 in HEK-Blue cell supernatants with standard P.g. LPS (0.1 μg/ml)
and simple solutions. Solutions were prepared as 100 nM of total zinc concentration
as stock solution and further diluted in the cell treatment

| | IL-8 Concentration (pg/ml) | | | | | |
|---|---|---|---|---|---|---|
| Sample ID | Replicate 1 | Replicate 2 | Replicate 3 | Replicate 4 | Average | Stdev |
| Medium | 417.95 | 376.95 | 295.95 | 277.55 | 342.1 | 57.6 |
| P.g. LPS | 2556.7 | 2606.85 | 2723.55 | 2748.55 | 2658.9 | 79.6 |
| P.g. LPS + ZnO 10 mM | 20.35 | 27.65 | 10.45 | 16.95 | 18.9 | 6.2 |
| P.g. LPS + ZnO 1 mM | 42.95 | 30.75 | 35.45 | 48.85 | 39.5 | 6.9 |
| P.g. LPS + ZnCitrate 10 mM | 11.85 | 11.85 | 3.15 | | 10.6 | 5.6 |
| P.g. LPS + ZnCitrate 1 mM | 55.35 | 27.95 | 26.65 | 29.55 | 34.9 | 11.9 |
| P.g. LPS + DZ 10 mM | 39.95 | 17.35 | 20.15 | 17.65 | 23.8 | 9.4 |
| P.g. LPS + DZ 1 mM | 32.15 | 34.15 | 38.95 | 38.35 | 35.9 | 2.8 |
| P.g. LPS + DZA 10 mM | 16.75 | 21.75 | 9.35 | 9.75 | 14.4 | 5.2 |
| P.g. LPS + DZA 1 mM | 37.15 | 37.85 | 54.55 | 62.55 | 48.0 | 10.9 |

The results suggested that with sufficient soluble zinc ion, the LPS-induced inflammation through binding to TLR4 receptor and further NF-kB activation was effectively blocked and pro-inflammatory cytokines were significantly suppressed.

In order to understand the role of Arginine in the inhibition of LPS induced inflammation, a dose response experiment was performed using ultrapure P.g. LPS, which only specifically targets TLR4 receptor, in order to maximize the activation of NF-kB and expression of IL-8.

The testing samples were prepared as the stock simple solutions. In order to easily compare the between the samples, the corresponding stock solutions of zinc oxide, zinc citrate, DZ and Arginine were prepared based on the DZA stock concentration as displayed in Table 6. The first serial dilution was made 3 times with water, a dilution factor simulating dilution of toothpastes while brushing. Additional serial dilutions were made by 2-fold of the first dilution to study dose response effect.

As shown in FIG. 6 viability of HEK-Blue cells co-incubated with each solution provided as described in Table. Viability of cells treated with Ultrapure P.g. LPS at 1 μg/mL was separately tested. Viability of untreated cells (UNT) is also shown.

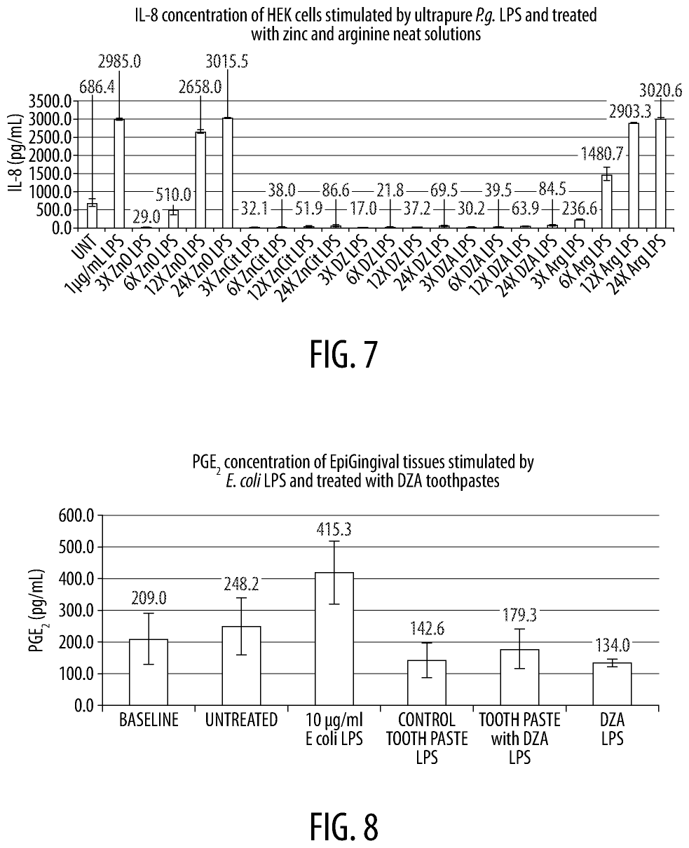

Date is shown in FIG. 7 and Table 8.

As shown in FIG. 7, at 3 times dilution of the stock simple solution, arginine and two zinc compounds individually demonstrated strong inhibition of IL-8 as well as the DZ and DZA. When dilution folds increase, zinc oxide and arginine lose the inhibition of IL-8 gradually.

TABLE 8
Concentration of IL-8 in HEK-Blue cell supernatants with standard
P.g. LPS (1 μg/ml) and simple solutions.

| | IL-8 Concentration (pg/ml) | | | | | |
|---|---|---|---|---|---|---|
| Sample ID | Replicate 1 | Replicate 2 | Replicate 3 | Replicate 4 | Average | Stdev |
| Medium | 862.0 | 623.3 | 618.5 | 641.8 | 686.4 | 101.8 |
| 1 μg P.g. LPS | 2993.5 | 3031.1 | 2942.3 | 2972.9 | 2985.0 | 32.3 |
| 3X ZnO | 32.6 | 25.5 | | | 29.0 | 3.6 |
| 6X ZnO | 646.1 | 374.0 | | | 510.0 | 136.1 |
| 12X ZnO | 2699.5 | 2616.4 | | | 2658.0 | 41.6 |
| 24X ZnO | 3010.4 | 3020.6 | | | 3015.5 | 5.1 |
| 3X ZnCitrate | 35.4 | 28.9 | | | 32.1 | 3.3 |
| 6X ZnCitrate | 38.1 | 37.9 | | | 38.0 | 0.1 |
| 12X ZnCitrate | 55.6 | 48.1 | | | 51.9 | 3.8 |
| 24X ZnCitrate | 88.5 | 84.8 | | | 86.6 | 1.8 |
| 3X DZ | 17.5 | 16.5 | | | 17.0 | 0.5 |
| 6X DZ | 23.3 | 20.2 | | | 21.8 | 1.5 |
| 12X DZ | 37.1 | 37.3 | | | 37.2 | 0.1 |
| 24X DZ | 71.6 | 67.4 | | | 69.5 | 2.1 |
| 3X DZA | 28.8 | 31.6 | | | 30.2 | 1.4 |
| 6X DZA | 42.6 | 36.4 | | | 39.5 | 3.1 |
| 12X DZA | 63.0 | 64.8 | | | 63.9 | 0.9 |
| 24X DZA | 94.7 | 74.2 | | | 84.5 | 10.3 |
| 3X Arg | 248.5 | 224.7 | | | 236.6 | 11.9 |
| 6X Arg | 1649.9 | 1311.5 | | | 1480.7 | 169.2 |
| 12X Arg | 2891.2 | 2915.5 | | | 2903.3 | 12.1 |
| 24X Arg | 3003.8 | 3037.4 | | | 3020.6 | 16.8 |

Sufficient soluble zinc ion, the LPS-induced inflammation. This confirmed that combining two zinc and arginine synergistically prevents the binding of P.g. LPS and TLR4 receptor, resulting in reduction of IL-8 expression in the cell supernatant.

### Example 5

Human Gingival 3D Tissue Treatment

Human Gingival tissues were treated with a dentifrice containing compositions comprising a combination of zinc citrate, zinc oxide and arginine and PGE 2 levels in the culture medium were quantified using ELISA kit.

MatTek Tissues

Human gingival tissue (MatTek EpiGingival tissues, MatTek Corporation, cat #Gin-100) were cultured in the culture medium supplied by the MatTek Corporation. Tissue was treated with toothpaste slurries at 1:2 dilution ratio with water or DZA solution topically for 2 min at room temperature. Standard E. coli LPS (Invivogen, cat #tlrl-eklps) was used to stimulate MatTek tissue as recommended by the manufacturer. E. coli LPS was prepared in the culture medium in a 6 well plate and co-incubate with treated tissues for 16 hr at 37° C.

Test Samples

Control toothpaste containing SnF₂+NaF+Zn phosphate

Toothpaste Containing DZA

DZA simple solution (1.5% L-arginine, 1.0% ZnO, 0.5% ZnCitrate trihydrate)

Procedure

MatTek EpiGingival tissues were removed from cold shipping and allowed to equilibrate two nights in 0.9 mL assay media (MatTek, cat #GIN-100-ASY) at 37° C. under 5% CO₂. Media was collected for baseline PGE₂ analysis. 100 ul of 1:2 dilution of control toothpaste, toothpaste containing DZA or DZA solution was applied to the top of tissues and treated for 2 minutes. The treatment was aspirated and the tissue washed twice with 200 μl sterile PBS. Tissues were incubated overnight in 1 mL assay media containing 10 μg/mL E. coli LPS or untreated media and incubated overnight. Media was collected the next day for analysis and stored at −20° C.

Sample Analysis

PGE₂ analysis was performed using the Enzo PGE 2 ELISA assay (Enzo Life Sciences, cat #ADI-900-001).

Results

Data is shown in FIG. 8 and Table 9.

As shown in FIG. 8, the toothpaste formulation containing DZA containing DZA demonstrated strong reduction of PGE₂ on 3D human gingival tissues similar performance of reducing PGE₂ as the simple solution DZA. This suggests the bioavailable of DZA was well maintained in the formulation. The other control formulation containing zinc phosphate and stannous fluoride also displayed strong anti-inflammatory efficacy, to the same extent as DZA formula and simple solution DZA.

TABLE 9
Concentration of PGE₂ in MatTek tissue supernatants with
Ecoli LPS (10 μg/ml) and tooth paste slurries or simple solutions.

| | PGE₂ concentmtion (pg/ml) | | | | |
|---|---|---|---|---|---|
| | | | Control | Toothpaste | |
| | Medium | Ecoli LPS | Toothpaste | with DZA | DZA |
| Replicate 1 | 208.7 | 505.2 | 146.1 | 224.5 | 144.8 |
| Replicate 2 | 202.2 | 485.3 | 160.4 | 246.5 | 150.6 |
| Replicate 3 | 172.4 | 298.7 | 70.4 | 203.5 | 113 |
| Replicate 4 | 168.4 | 245.2 | 75.9 | 217.8 | 118.3 |
| Replicate 5 | 364.6 | 479.3 | 206.8 | 94.6 | 131.8 |
| Replicate 6 | 373 | 478 | 196.1 | 88.7 | 145.2 |
| Average | 248.2 | 415.3 | 142.6 | 179.3 | 134.0 |
| STD | 86.5 | 102.9 | 53.2 | 63.3 | 14.2 |

Conclusions

This study and those described in the examples above, suggests that formulations containing zinc oxide, zinc citrate and arginine display strong anti-inflammatory efficacy in the presence of pathogenic bacterial endotoxins.

### Example 6

Oral compositions that comprise arginine are disclosed in WO 2014/088572, which corresponds to US 2015/0313813, which are both incorporated herein by reference. In some embodiments the oral care composition comprises: from about 0.05 to about 5% by weight, of a combination of zinc citrate and zinc oxide; a fluoride ion source in an amount effective to deliver from about 500 to about 5,000 ppm fluoride, and from about 0.1 to about 10%, by weight, of arginine. In some such embodiments, the oral composition is in the form of a dentifrice comprising an abrasive. In some such embodiments, the amount of zinc is 0.5 to 4% by weight. In some such embodiments, the compositions may further comprise one or more abrasives, one or more humectants, and one or more surfactants. In some such embodiments, the compositions may further comprise an effective amount of one or more alkali phosphate salts and/or an effective amount of one or more antibacterial agents and/or a whitening agent. In some such embodiments, the composition comprises zinc phosphate and one or more other sources of zinc ion. In some such embodiments, the pH of the composition is basic. In some such embodiments, the composition may comprise, in a silica abrasive dentifrice base: 1 to 3% zinc citrate; 1 to 8% arginine; 700 to 2000 ppm fluoride; and 2 to 8% alkali phosphate salts selected from sodium phosphate dibasic, potassium phosphate dibasic, dicalcium phosphate dihydrate, tetrasodium pyrophosphate, tetrapotassium pyrophosphate, calcium pyrophosphate, sodium tripolyphosphate, and a combination of two or more thereof.

### Example 7

Oral compositions that comprise arginine are disclosed in WO 2015/094849, which corresponds to US 2016/0338921, which are both incorporated herein by reference. In some embodiments the oral care composition comprises: arginine, in free or salt form; and zinc oxide and zinc citrate. In some embodiments, the arginine is present in an amount of 0.5 weight % to 3 weight %, such as 1 weight % to 2.85 weight %, such as 1.17 weight % to 2.25 weight %, such as 1.4 weight % to 1.6 weight %, such as about 1.5 weight %, based on the total weight of the composition. In some embodiments set out above, the total concentration of zinc salts in the composition is 0.2 weight % to 5 weight %, based on the total weight of the composition. In some embodiments set out above, the molar ratio of arginine to total zinc salts is 0.05:1 to 10:1. In some embodiments set out above, the composition comprises zinc oxide in an amount of 0.5 weight % to 1.5 weight %, such as 1 weight %, and zinc citrate in an amount of 0.25 weight % to 0.75 weight %, such as 0.5 weight %, based on the total weight of the composition. In some embodiments set out above, the weight ratio of zinc oxide to zinc citrate is 1.5:1 to 4.5:1, optionally 1.5:1 to 4:1, 1.7:1 to 2.3:1, 1.9:1 to 2.1:1, or about 2:1.

### Example 8

Oral compositions that comprise arginine are disclosed in WO 2017/003844, which corresponds to US 2018/0021234, which are both incorporated herein by reference. In some embodiments, the oral care composition comprises: arginine, zinc oxide and zinc citrate and a fluoride source. In some embodiments, the arginine has the L-configuration. In some embodiments, the arginine is present in an amount corresponding to 0.1% to 15%, or 0.1% to 8%, or about 5.0 wt. %, or about 8.0 wt. %, or about 1.5 wt. %, based on the total weight of the composition, the weight of the arginine acid being calculated as free form. In some embodiments, the arginine is in free form or partially or wholly salt form. In some embodiments set out above, the ratio of the amount of zinc oxide (by wt %) to zinc citrate (by wt %) is 2:1, 2.5:1, 3:1, 3.5:1 or 4:1, wherein the ratio is by wt. of the overall composition. In some embodiments, the zinc citrate is in an amount of from 0.25 to 1.0 wt % and zinc oxide may be present in an amount of from 0.75 to 1.25 wt % or the zinc citrate is in an amount of about 0.5 wt % and zinc oxide is present in an amount of about 1.0%, based on the total weight of the composition. In some embodiments set out above, the fluoride source is sodium fluoride or sodium monofluorophosphate. In some such embodiments, the sodium fluoride or sodium monofluorophosphate is from 0.1 wt. %-2 wt. % based on the total weight of the composition. In some embodiments, the sodium fluoride or sodium monofluorophosphate is a soluble fluoride salt which provides soluble fluoride in amount of 50 to 25,000 ppm fluoride, such as in an amount of about 1000 ppm-1500 ppm, for example in an amount of about 1450 ppm. In some embodiments the fluoride source is sodium fluoride in an amount about 0.32% by wt, based on the total weight of the composition. In some embodiments, the fluoride source is stannous fluoride. Some embodiments set out above further comprise a preservative selected from: benzyl alcohol, Methylisothizolinone (“MIT”), Sodium bicarbonate, sodium methyl cocoyl taurate (tauranol), lauryl alcohol, and polyphosphate. Some embodiments set out above further comprise benzyl alcohol in an amount of from 0.1-0.8% wt %, or from 0.3-0.5% wt %, or about 0.4 wt % based on the total weight of the composition. In some embodiments, the oral care composition comprises about 1.0% zinc oxide, about 0.5% zinc citrate, about 1.5% L-arginine, about 1450 ppm sodium fluoride, and optionally about benzyl alcohol 0.1 wt. % and/or about 5% small particle silica (e.g., AC43), based on the total weight of the composition. In some embodiments, the oral care composition comprises about 1.0% zinc oxide, about 0.5% zinc citrate, about 5% L-arginine, about 1450 ppm sodium fluoride, and optionally about benzyl alcohol 0.1 wt. % and/or about 5% small particle silica (e.g., AC43), based on the total weight of the composition. In some embodiments set out above, the oral care composition may comprise about 1.0% zinc oxide, about 0.5% zinc citrate, about 1.5% L-arginine, about 0.22%-0.32% sodium fluoride, about 0.5% tetrasodium pyrophosphate, and optionally about benzyl alcohol 0.1 wt. %, based on the total weight of the composition. In some embodiments set out above, the oral care composition may be any of the following oral care compositions selected from the group consisting of: a toothpaste or a dentifrice, a mouthwash or a mouth rinse, a topical oral gel, and a denture cleanser.

### Example 9

Oral compositions that comprise arginine are disclosed in WO 2017/003856, which is incorporated herein by reference. In some embodiments, the oral care composition comprises: arginine in free or salt form, zinc oxide and zinc citrate and a fluoride source comprising stannous fluoride. In some embodiments, the arginine has the L-configuration. In some embodiments, the arginine is present in an amount corresponding to 0.1% to 15%, or 0.1% to 8%, or about 5.0 wt. %, or about 8.0 wt. %, or about 1.5 wt. %, based on the total weight of the composition, the weight of the arginine acid being calculated as free form. In some embodiments, the arginine is in free form or partially or wholly in salt form. In some embodiments set out above, the ratio of the amount of zinc oxide (by wt. %) to zinc citrate (by wt. %) is 2:1, 2.5:1, 3:1, 3.5:1 or 4:1, wherein the ratio is by weight of the overall composition. In some embodiments set out above, the zinc citrate is in an amount of from 0.25 to 1.0 wt. % and zinc oxide may be present in an amount of from 0.75 to 1.25 wt. % or the zinc citrate is in an amount of about 0.5 wt. % and zinc oxide is present in an amount of about 1.0 wt. %, based on the total weight of the composition. In some embodiments set out above, the fluoride source further comprises at least one member selected from the group of: sodium fluoride, potassium fluoride, sodium monofluorophosphate, sodium fluorosilicate, ammonium fluorosilicate, amine fluoride (e.g., N′-octadecyltrimethylendiamine-N,N,N′-tris(2-ethanol)-dihydrofluoride), ammonium fluoride, titanium fluoride, hexafluorosulfate, and combinations thereof. In some embodiments set out above, the stannous fluoride is present in an amount from 0.1 wt. % to 2 wt. % based on the total weight of the composition. In some embodiments set out above, the stannous fluoride is a soluble fluoride salt which provides soluble fluoride in amount of 50 to 25,000 ppm fluoride, or about 750-7000 ppm, or about 1000-5500 ppm, or about 5000 ppm. In some embodiments, the oral care composition comprises about 1.0% zinc oxide, about 0.5% zinc citrate, about 1.5% L-arginine, about 750-7000 ppm fluoride; and optionally, about 5% small particle silica (e.g., AC43), based on the total weight of the composition. In some embodiments, the oral care composition comprises about 1.0% zinc oxide, about 0.5% zinc citrate, about 750-7000 ppm stannous fluoride; and optionally about 39.2% glycerin based on the total weight of the composition. In some embodiments set out above, the oral care composition may comprise about 1.0% zinc oxide, about 0.5% zinc citrate, about 1.5% L-arginine, stannous fluoride, and optionally about benzyl alcohol 0.1 wt. %, based on the total weight of the composition. In some embodiments set out above, the oral care composition may be any of the following oral compositions selected from the group consisting of: a toothpaste or a dentifrice, a mouthwash or a mouth rinse, a topical oral gel, and a denture cleanser.

### Example 10

Oral compositions that comprise arginine are disclosed in WO 2017/223169, which is incorporated herein by reference. In some embodiments, the oral care composition comprises: arginine in free or salt form, zinc oxide and zinc citrate and a fluoride source comprising stannous fluoride. In some embodiments, the oral care compositions comprise zingerone, zinc oxide, zinc citrate; and a stannous fluoride. In some embodiments, the zingerone is present in an amount of from 0.01% to 1%, based on the total weight of the composition. In some embodiments, the ratio of the amount of zinc oxide (by wt %) to zinc citrate (by wt %) is 2:1, 2.5:1, 3:1, 3.5:1 or 4:1, based on the total weight of the composition. In some embodiments, the zinc citrate is present in an amount of from 0.25 to 1.0 wt % and zinc oxide is present in an amount of from 0.75 to 1.25 wt %, based on the total weight of the composition. In some embodiments, the zinc citrate is present in an amount of about 0.5 wt % and zinc is present in an amount of about 1.0% based on the total weight of the composition. In some embodiments, the stannous fluoride is present in an amount of 0.1 wt, % to 2 wt. %, based on the total weight of the composition. Some embodiments further comprise synthetic amorphous precipitated abrasive silica in an amount of from 1%-25% by wt, based on the total weight of the composition and/or a high cleaning silica in an amount of from 1 wt %-15 wt %, based on the total weight of the composition. Some embodiments further comprise an effective amount of one or more alkali phosphate salts, for example sodium tripolyphosphate in an amount of from 1-5 wt %, based on the total weight of the composition. Some embodiments further comprise citric acid in an amount of from 0.1-3 wt. %, and citrate ion, for example trisodium citrate dihydrate, in an amount of from 0.1-5 wt. %, based on the total weight of the composition. Some embodiments further comprise carboxymethyl cellulose in an amount of from 0.1 wt, %-1.5 wt. %, based on the total weight of the composition. Some embodiments further comprise an anionic surfactant, e.g., sodium lauryl sulfate, in an amount of from 0.5-5% by weight, based on the total weight of the composition. Some embodiments further comprise an amphoteric surfactant in an amount of from 0.5-5%, based on the total weight of the composition. Some embodiments further comprise a PVM/MA copolymer, such as for example a Gantrez polymer, in an amount of from 0.1-5 wt. %, based on the total weight of the composition. Some embodiments further comprise microcrystalline cellulose/sodium carboxymethylcellulose. Some embodiments further comprise one or both of polyethylene glycol in an amount of from 1-6%; and propylene glycol in an amount of from 1-6%, based on the total weight of the composition. Some embodiments further comprise polyvinylpyrrolidone (PVP) in an amount of from 0.5-3 wt. %, based on the total weight of the composition. Some embodiments further comprise from 5%-40% free water by weight, based on the total weight of the composition. Some embodiments further comprise one or more thickening agents, e.g. sodium carboxymethyl cellulose and sodium carboxy methyl hydroxyethyl cellulose,

In some embodiments, the oral care composition comprises: about 0.1-0.3% zingerone; about 1.0% zinc oxide; about 0.5% zinc citrate, and about 0.4%-0.5% stannous fluoride.

In some embodiments, the oral care composition comprises: about 0.1-0.3% zingerone; about 1.0% zinc oxide; about 0.5% zinc citrate, about 0.4%-0.5% stannous fluoride; and about 1.2% abrasive silica and may, in some such embodiments, further comprise about 7% wt % high cleaning silica, based on the total weight of the composition, and/or a surfactant system comprising one or both of an anionic surfactant in an amount of from 0.5-5%, by weight; and/or an amphoteric surfactant in an amount of from 0.5-5% by weight, based on the total weight of the composition. Some embodiments further comprise sodium tripolyphosphate in an amount of from 1-5 wt %, based on the total weight of the composition and/or sodium phosphate in an amount of from 0.5 wt %-5 wt %, based on the total weight of the composition. Examples of the oral composition include a toothpaste or a dentifrice, a mouthwash or a mouth rinse, a topical oral gel, a chewing gum, or a denture cleanser.

### Example 11

Test dentifrices comprising arginine, zinc oxide, zinc citrate and a source of fluoride were prepared as shown in Tables A-E:

TABLE A

| Ingredient | Compound I |
|---|---|
| Humectants | 20.0-25.0 |
| Non-ionic surfactant | 1.0-2.0 |
| Amphoteric stufactant | 3.0-4.0 |
| Flavoring/fragrance/coloring agent | 2.0-3.0 |
| Polymers | 10.0-15.0 |
| pH adjusting agents | 1.5-3.0 |
| Precipitated Calcium Carbonate | 35 |
| Zinc citrate trihydrate | 0.5 |
| Zinc oxide | 1.0 |
| Sodium Fluoride - USP, EP | 0.32 |
| Arginine Bicatbonate | 13.86 |
| Demineralized water | QS |

TABLE B

| Ingredient | Compound A | Compound B | Compound C | Compound D |
|---|---|---|---|---|
| Humectants | 25.0-40.0 | 25.0-40.0 | 25.0-40.0 | 25.0-40.0 |
| Anionic surfactant | 1.0-3.0 | 1.0-3.0 | 1.0-3.0 | 1.0-3.0 |
| Flavoring/fragrance/coloring agent | 2.5-4.0 | 2.5-4.0 | 2.5-4.0 | 2.5-4.0 |
| Polymers | 4.0-6.0 | 4.0-6.0 | 4.0-6.0 | 4.0-6.0 |
| pH adjusting agents | 5.0-6.0 | 5.0-6.0 | 5.0-6.0 | 5.0-6.0 |
| Synthetic Amorphous | 16.00 | 21.37 | 17.92 | 7.81 |
| Precipitated Silica | | | | |
| Alumina | 0.02 | 0.01 | 0.01 | 0.01 |
| Silica | — | — | — | 15.0 |
| Laniyl alcohol | 0.02 | 0.02 | 0.02 | 0.02 |
| Zinc citrate | 0.5 | 0.5 | 0.5 | 0.5 |
| Zinc oxide | 1.0 | 1.0 | 1.0 | 1.0 |
| Sodium Fluoride - USP, EP | 0.32 | 0.32 | 0.32 | 0.32 |
| L-Arginine Bicarbonate | 5.0 | 5.0 | 5.0 | 5.0 |
| Demineralized water | QS | QS | QS | QS |

TABLE C

| Ingredient | Compound E | Compound F | Compound G |
|---|---|---|---|
| Humectants | 25.0-40.0 | 25.0-40.0 | 25.0-40.0 |
| Anionic surfactant | 1.0-3.0 | 1.0-3.0 | 1.0-3.0 |
| Non-ionic surfactant | 0.1-1.0 | 0.1-1.0 | 0.1-1.0 |
| Amphoteric surfactant | 0.1-1.0 | 0.1-4.0 | 0.1-1.0 |
| Flavoring/fragrance/ | 4.0-6.0 | 4.0-6.0 | 4.0-6.0 |
| coloring agent | | | |
| Polymers | 0.1-2.0 | 0.1-2.0 | 0.1-2.0 |
| pH adjusting agents | 5.0-6.0 | 5.0-6.0 | 5.0-6.0 |
| Thickener | 6.0 | 6.5 | 7,0 |
| Alumina | 0.1 | 0.1 | 0.1 |
| Synthetic Amorphous | 17.6 | 8.8 | 22.4 |
| Precipitated Silica | | | |
| Silica | — | 15.0 | — |
| Benzyl alcohol | 0.1 | 0.1 | 0.1 |
| Synthetic Amorphous Silica | 5.0 | 5.0 | 5.0 |
| Zinc citrate | 0.5 | 0.3 | 0.5 |
| Zinc oxide | 1.0 | 1.0 | 1.0 |
| Sodium Fluoride - USP, EP | 0.32 | 0.32 | 0.32 |
| L-Arginine Bicarbonate | 1.5 | 1.5 | 1.5 |
| Demineralized water | QS | QS | QS |

TABLE D

| Ingredient | Compound H | Compound I |
|---|---|---|
| Humectants | 45.0-55.0 | 35.0-45.0 |
| Abrasives | 14.0-16.0 | 9.0-11.0 |
| Anionic surfactant | 1.0-3.0 | 1.0-3.0 |
| Non-ionic surfactant | 0.1-1.0 | — |
| Amphoteric surfactant | 1.0-7.0 | — |
| Flavoring/fragrance/coloring agent | 1.0-3.0 | 2.0-4.0 |
| Polymers | 0.1-2.0 | 3.0-8.0 |
| pH adjusting agents | 0.1-2.0 | 4.0-8.0 |
| Silica Thickener | 5.0 | 5.0-10.0 |
| Benzyl alcohol | 0.1 | — |
| Zinc citrate trihydrate | 0.5 | 0.5 |
| Zinc oxide | 1.0 | 1.0 |
| Sodium Fluoride - USP, EP | 0.32 | 0.32 |
| L-Arginine | 1.5 | 5.0 |
| Demineralized water | QS | QS |

TABLE E

| Ingredient | Compound I | Compound K | Compound L |
|---|---|---|---|
| Humectants | 20.0-50.0 | 20.0-50.0 | 20.0-50.0 |
| Abrasives | 5.0-20.0 | 5.0-20.0 | 5.0-20.0 |
| Anionic surfactant | 1.0-3.0 | 1.0-3.0 | 1.0-3.0 |
| Non-ionic surfactant | 0.1-1.0 | 0.1-1.0 | 0.1-1.0 |
| Amphoteric surfactant | 0.1-2.0 | 0.1-2.0 | 0.1-2.0 |
| Flavoring/fragrance/ | 1.0-5.0 | 1.0-5.0 | 1.0-5.0 |
| coloring agent | | | |
| Polymers | 0.1-2.0 | 0.1-2.0 | 0.1-2.0 |
| PH adjusting agents | 0.1-2.0 | 0.1-2.0 | 0.1-2.0 |
| Thickener | 6.0 | 6.5 | 7.0 |
| Dental type silica | — | — | 15.0 |
| High cleaning silica | — | 15.0 | — |
| Synthetic Abrasives | 10.0 | — | — |
| Synthetic Amorphous Silica | 5.0 | 5.0 | 5.0 |
| Benzyl alcohol | 0.4 | 0.4 | 0.4 |
| Zinc citrate trihydrate | 0.5 | 0.5 | 0.5 |
| Zinc oxide | 1.0 | 1.0 | 1.0 |
| Sodium Fluoride - USP, EP | 0.32 | 0.32 | 0.32 |
| L-Arginine | 1.5 | 1.5 | 1.5 |
| Demineralized water | QS | QS | QS |

### Example 12

Test dentifrices comprising arginine, zinc oxide, zinc citrate and stannous fluoride were prepared as shown in Table F:

TABLE F

| Ingredient | | | |
|---|---|---|---|
| Humectants | 20.0-60.0 | 20.0-50.0 | 20.0-50.0 |
| Abrasives | 10.0-40.0 | 5.0-20.0 | 5.0-20.0 |
| Anionic surfactant | 1.0-3.0 | 1.0-3.0 | 1.0-3.0 |
| Amphoteric surfactant | 0.5-1.5 | 0.1-2.0 | 0.1-2.0 |
| Flavoring/fragrance/coloring agent | 0.5-5.0 | 1.0-5.0 | 1.0-5.0 |
| Polymers | 1.0-10.0 | 0.1-2.0 | 0.1-2.0 |
| pH adjusting agents | 1.0-10.0 | 0.1-2.0 | 0.1-2.0 |
| Zinc citrate | 0.25-1.0 | 0.5 | 0.5 |
| Zinc oxide | 0.75-1.25 | 1.0 | 1.0 |
| Stannous Fluoride | 0.1-1.0 | 0.32 | 0.32 |
| L-Arginine | 0.1-10.0 | 1.5 | 1.5 |
| Demineralized water | QS | QS | QS |

### Example 13

Test dentifrices comprising arginine, zinc oxide, zinc citrate and stannous fluoride were prepared as shown in Table G:

TABLE G

| Ingredient | | | |
|---|---|---|---|
| Demineralized water | 8.8 | 8.8 | 88 |
| Sodium Saccharin | 0.8 | 0.8 | 0.8 |
| Trisodium citrate dihydrate | 30 | 30 | 3.0 |
| Citric acid anhydrous | 0.6 | 0.6 | 0.6 |
| Stannous fluoride | 0.454 | 0.454 | 0.454 |
| Zinc oxide | 1.0 | 1.0 | 1.0 |
| 99.0-101.0% glycerin | 40.9 | 40.9 | 40.9 |
| Polyethylene glycol | 3.0 | 3.0 | 3.0 |
| Propylene glycol | 4.0 | 4.0 | 4.0 |
| Thickeners (including xanthan gum, | 1.4 | 1.4 | 1.4 |
| carboxymethyl cellulose, | | | |
| microcrystalline cellulose NaCMV) | | | |
| PVP | 1.25 | 1.25 | 1.25 |
| Dye | 0.002 | 0.002 | 0.002 |
| Abrasives (including synthetic amorphous silica, | 24.0 | 24.0 | 240 |
| precipitated silica; high cleaning silica, silicon dioxide) | | | |
| Sodium lauryl sulfate powder | 1.75 | 1.75 | 1.75 |
| Cocamidopropyl betaine (30% solution) | 1.0 | 1.0 | 11.0 |
| Gantrez S-97 (16.5% solution) | 0.606 | 0.606 | 0.606 |
| Titanium dioxide coated mica | 0.115 | 0.115 | 0.115 |
| 85% syrupy phosphoric acid food grade | 0.60 | 0.60 | 0.60 |
| Sodium triphosphate tribasic 12-hydrate | 1.0 | 1.0 | 1.0 |
| Zinc citrate trihydrate | 0.5 | 0.5 | 0.5 |
| Sodium tripolyphosphate | 3.0 | 3.0 | 3.0 |
| FCC grade flavor | 2.2 | 2.1 | 1.9 |
| zingerone | — | 0.10 | 0.30 |

1. A method for identifying a composition that neutralizes toxicity of a lipopolysaccharide comprising:
performing a test assay that comprises the steps of:
contacting a Toll-like receptor reporter cell with an amount of a lipopolysaccharide sufficient to stimulate secretion of one or more proinflammatory cytokines by the Toll-like receptor reporter cell and a test composition; and
measuring the amount of one or more proinflammatory cytokines and/or prostaglandin E2 secreted by the Toll receptor reporter cell in the test assay; and
performing a control assay that comprises the steps of:
in the absence of the test composition, contacting the Toll-like receptor reporter cell with the amount of the lipopolysaccharide sufficient to stimulate secretion of one or more proinflammatory cytokines and/or prostaglandin E2 by the Toll-like receptor reporter cell; and
measuring the amount of one or more proinflammatory cytokines and/or prostaglandin E2 secreted by the Toll-like receptor reporter cell in the control assay.
comparing the amount of one or more proinflammatory cytokines and/or prostaglandin E2 secreted by the Toll-like receptor reporter cell in the test assay with the amount of one or more proinflammatory cytokines and/or prostaglandin E2 secreted by the Toll-like receptor reporter cell in the control assay;
wherein if the amount of one or more proinflammatory cytokines and/or prostaglandin E2 secreted by the Toll-like receptor reporter cell in the test assay is less than the amount of one or more proinflammatory cytokines and/or prostaglandin E2 secreted by the Toll-like receptor reporter cell in the control assay, the test composition is identified as a composition that neutralizes toxicity of a lipopolysaccharide.

2. The method of claim 1 wherein the Toll-like receptor reporter cell is selected from the group consisting of: a recombinant HEK 293T cell that expresses one or more Toll-like receptors, a recombinant human monocyte cell that expresses one or more Toll-like receptors, and a recombinant Chinese Hamster ovary cell that expresses one or more Toll-like receptors.

3. The method of claim 1 wherein the Toll-like receptor reporter cell is a recombinant HEK 293T cell that expresses one or more Toll-like receptors.

4. The method of claim 1 wherein the Toll-like receptor reporter cell is HEK 293T TLR4.

5. The method of claim 1 wherein the Toll-like receptor reporter cell is a human gingival cell that is maintained in vitro as part of cultured human gingival tissue, wherein the human gingival cell expresses one or more Toll-like receptors.

6. The method of claim 1 wherein the Toll-like receptor reporter cell expresses one or more Toll-like receptors selected from the group consisting of: TLR1, TLR2, TLR3, TLR4, TLR5, TLR6, TLR7, TLR8, TLR9 and TLR10.

7. The method of claim 1 wherein the Toll-like receptor reporter cell expresses TLR2, and TLR4.

8. The method of claim 1 wherein the Toll-like receptor reporter cell expresses TLR4.

9. The method of claim 1 wherein the lipopolysaccharide is from a species of bacteria selected from the group consisting of: Porphyromonas gingivalis, Escherichia coli, Prevotella intermedia, Fusobacterium nucleatum, Treponema denticola, Aggregatibacter actinomycetemcomitans and Tannerella forsythia.

10. The method of claim 1 wherein the lipopolysaccharide is from Porphyromonas gingivalis.

11. The method of claim 1 wherein the amount of the one or more proinflammatory cytokines secreted by the Toll-like receptor reporter cell is measured.

12. The method of claim 11 wherein the amount of one or more proinflammatory cytokines secreted by the Toll-like receptor reporter cell is measured using proinflammatory cytokine-specific magnetic beads.

13. The method of claim 1 wherein the amount of the one or more proinflammatory cytokines secreted by the Toll-like receptor reporter cell is measured and the one or more proinflammatory cytokines is selected from the group consisting of: TNF-α, IL-6, IL-8, IL-1β and GM-CSF.

14. The method of claim 13 wherein the amount of one or more proinflammatory cytokines secreted by the Toll-like receptor reporter cell is measured using proinflammatory cytokine-specific magnetic beads.

15. The method of claim 1 wherein the amount of IL-8 secreted by the Toll-like receptor reporter cell is measured.

16. The method of any of claim 15 wherein the amount of IL-8 secreted by the Toll-like receptor reporter cell is measured using IL-8-specific magnetic beads.

17. The method of claim 1 wherein the amounts of two or more proinflammatory cytokines secreted by the Toll-like receptor reporter cell are measured.

18. The method of claim 17 wherein the amounts of two or more proinflammatory cytokines secreted by the Toll-like receptor reporter cell are measured, and the two or more proinflammatory cytokines are selected from the group consisting of: TNF-α, IL-6, TL-8, IL-1β and GM-CSF.

19. The method of claim 18 wherein the amount of two or more proinflammatory cytokines secreted by the Toll-like receptor reporter cell is measured using proinflammatory cytokine-specific magnetic beads.

20. The method of claim 19 wherein the amount of each of TNF-α, TL-6, TL-8, IL-1β and GM-CSF secreted by the Toll-like receptor reporter cell is measured.

21. The method of claim 20 wherein the amount of each of TNF-α, TL-6, TL-8, TL-1β and GM-CSF secreted by the Toll-like receptor reporter cell is measured using a panel of proinflammatory cytokine-specific magnetic beads that comprises TNF-α-specific magnetic beads, IL-6-specific magnetic beads, IL-8-specific magnetic beads, IL-1β-specific magnetic beads and GM-CSF-specific magnetic beads.

22. The method of claim 1 wherein the amounts Prostaglandin E2 secreted by the Toll-like receptor reporter cell are measured.

23. The method of claim 22 wherein the Toll-like receptor reporter cell is a human gingival cell that is maintained in vitro as part of cultured human gingival tissue, wherein the human gingival cell expresses one or more Toll-like receptors.

24. The method of claim 1 wherein two or more test assays are performed using a series of concentrations of the test composition.

25. The method of claim 1 wherein two or more test assays are performed using a series of concentrations of the lipopolysaccharide and two or more control assays are performed using the series of concentrations of the lipopolysaccharide.

26. A method of neutralizing toxicity of a lipopolysaccharide in an individual's oral cavity comprising administering to the oral cavity of the individual an oral care composition comprising a composition identified as a composition that neutralizes toxicity of the lipopolysaccharide by the method of claim 1, the method comprising applying the composition to the individual's oral cavity in an amount effective to inhibit secretion of one or more proinflammatory cytokines and/or prostaglandin E2 by cells of the individual.

27. The method of claim 26 wherein the oral care composition is a toothpaste.

28. A method of neutralizing toxicity of a lipopolysaccharide in an individual's oral cavity comprising administering to the oral cavity of the individual an oral care composition comprising zinc oxide and zinc citrate, and optionally, fluoride and/or arginine, in an amount effective to inhibit secretion of one or more proinflammatory cytokines and/or prostaglandin E2 by cells of the individual.

29. The method of claim 28 wherein the oral care composition is a toothpaste.

30. The method of claim 28 wherein:
the zinc oxide is present in an amount of from 0.75 to 1.25 wt % based on the total weight of the composition,
the zinc citrate is present in an amount of from 0.25 to 1.0 wt % based on the total weight of the composition.

31. The method of claim 28 wherein the ratio of the amount of zinc oxide by wt % to zinc citrate by wt % is 2:1, 2.5:1, 3:1, 3.5:1 or 4:1, based on the total weight of the composition.

32. The method of claim 28 wherein the ratio of the amount of zinc oxide by wt % to zinc citrate by wt % is about 2:1, based on the total weight of the composition.

33. The method of claim 28 wherein arginine is present in an amount of from 0.1% to 15%, based on the total weight of the composition, the weight of the basic amino acid being calculated as free form.

34. The method of claim 28 wherein arginine is present in an amount of from 0.5% to 3%, based on the total weight of the composition, the weight of the basic amino acid being calculated as free form.

35. The method of claim 33 wherein the arginine is L-arginine.

36. The method of claim 33 wherein the arginine is in free form.

37. The method of claim 33 wherein the arginine is in salt form.

38. The method of claim 28 wherein the oral care composition comprises stannous fluoride.

39. The method of claim 38 wherein the oral care composition comprises stannous fluoride in an amount of 0.1 wt, % to 2 wt. % based on the total weight of the composition.

40. The method of claim 28 wherein the individual is identified as having inflammation of tissue their oral cavity.

41. The method of claim 28 wherein the individual is identified as having inflammation of tissue their oral cavity caused by a pro-inflammatory response stimulated by toxicity of a lipopolysaccharide in the oral cavity.

42. The method of claim 28 wherein the individual is identified as having plaque and inflammation in the oral cavity.

43. The method of claim 28 wherein the individual is identified as having plaque and inflammation within an individual's gingival crevice.

44. The method of claim 28 wherein the individual is identified as having plaque which comprises gram negative bacteria and inflammation in the oral cavity.

45. The method of claim 28 wherein the individual is identified as having plaque which comprises gram negative bacteria and inflammation within an individual's gingival crevice.

46. The method of claim 28 wherein the individual is identified as having plaque which comprises gram negative bacteria, wherein the gram-negative bacteria are selected from the group consisting of Porphyromonas gingivalis, Escherichia coli, Prevotella intermedia, Fusobacterium nucleatum, Treponema denticola, Aggregatibacter actinomycetemcomitans and Tannerella forsythia.

47. The method of claim 28 wherein the individual is identified as having plaque which comprises Porphyromonas gingivalis.
